(19) United States
(12) Patent Application Publication
(10) Pub. No.: US 2021/0171919 A1
(43) Pub. Date: Jun. 10, 2021

(54) LUCIFERASE VARIANT

(71) Applicant: Kikkoman Corporation, Chiba (JP)

(72) Inventors: Kanako HAYASHI, Chiba (JP); Atsushi ICHIYANAGI, Chiba (JP)

(73) Assignee: Kikkoman Corporation, Chiba (JP)

(21) Appl. No.: 17/257,745

(22) PCT Filed: Jul. 5, 2019
(86) PCT No.: PCT/JP2019/026784

§ 371 Date: Jan. 10, 2021

(30) Foreign Application Priority Data
Jul. 6, 2018 (JP) 2018-129384

(51) Int. Cl. C12N 9/02; C12Q 1/66

(52) U.S. Cl. CPC C12N 9/0069; C12Y 113/12007; C12Q 1/66

(57) ABSTRACT

In one embodiment, an object of the present invention is to provide a firefly luciferase having improved thermostability. In one embodiment, the present invention relates to a luciferase mutant having improved thermostability that is a mutant of firefly luciferase comprising an amino acid sequence in which the amino acid residue at the position corresponding to position 393 of SEQ ID NO 1 is substituted, a polynucleotide encoding the luciferase mutant, and a production method of the luciferase mutant.

### TECHNICAL FIELD

The present invention relates to luciferase mutants having improved thermostability, polynucleotides encoding the luciferase mutants, production methods of the luciferase mutants, kits for detecting at least one of ATP, ADP, or AMP comprising the luciferase mutants, methods for detecting at least one of ATP, ADP, or AMP comprising using the luciferase mutant, and the like.

### BACKGROUND ART

Firefly luciferase is an enzyme that converts adenosine triphosphate (ATP), D-luciferin, and oxygen into adenosine monophosphate (AMP), oxyluciferin, and carbon dioxide, in the presence of magnesium ions and oxygen, thus generating light. Applying the luminous principle of firefly luciferase allows measuring trace amounts of an enzyme reaction substrate with significantly high sensitivity. Therefore, firefly luciferase has been widely used, for example, for detection of microorganisms in food and beverage materials, assessment of food residue and contamination adhering to fingers and implements, or high-sensitivity measurement methods using various kinds of antibody techniques and gene amplification techniques, or the like, using ATP as the indicator.

However, since coleoptera luciferases, such as firefly luciferase, are generally unstable to heat, there is a drawback that coleoptera luciferases are susceptible to inactivation when being stored as reagents. Thus, attempts have been made to obtain a luciferase that overcomes this drawback and has satisfactory thermostability.

One attempt is to devise the formulation by adding a salt or the like to the measuring reagent to ensure stability to some extent. However, this method cannot be widely applied due to, for example, constraints of the reagent composition, and has a drawback that the addition of salts is likely to cause some type of reaction impediment in the luciferase reaction.

One of the different attempted approaches besides the devising of the reagent composition is to search for mutant luciferases having preferred properties. For example, Non Patent Literature 1 reports that a North American firefly (Photinus pyralis) luciferase in which the amino acid at position 342 is mutated to alanine is obtained and the luminescence persistency of this firefly luciferase is improved. Additionally, Patent Literature 1 discloses that luciferase of Genji firefly (Luciola cruciata) or Heike firefly (Luciola lateralis) in which the amino acid at position 217 is substituted with a hydrophobic amino acid has heat resistance. Patent Literature 2 discloses that a firefly luciferase having an amino acid sequence in which the amino acid equivalent to position 287 of Heike firefly luciferase is mutated to alanine or the amino acid equivalent to position 392 of Heike firefly luciferase is mutated to isoleucine has improved thermostability.

However, the luciferases having the substitutions disclosed in these documents did not necessarily have sufficient thermostability.

### CITATION LIST

### Patent Literature

Patent Literature 1: JP 1105-244942 A (1993)
Patent Literature 2: JP 2011-120559 A

### Non Patent Literature

Non Patent Literature 1: Biochemistry 2003, No. 42, p10429-10436

### SUMMARY OF INVENTION

### Technical Problem

An object of the present invention is to provide firefly luciferase having improved thermostability.

### Solution to Problem

The inventor has found that a mutation that substitutes cysteine at position 393 of Luciola lateralis with a non-acidic amino acid other than cysteine improves thermostability of the firefly luciferase. Additionally, the inventor has found that a substitution of the amino acid at the position corresponding to position 393 of SEQ ID NO 1 can improve thermostability of firefly luciferase, thus having completed the present invention.

Accordingly, the present invention encompasses the following aspects.

(1) A luciferase mutant having improved thermostability wherein the luciferase mutant is a mutant of firefly luciferase, said mutant comprising an amino acid sequence in which the amino acid residue at the position corresponding to position 393 of SEQ ID NO 1 is substituted.
(2) A luciferase mutant having improved thermostability wherein the luciferase mutant is a mutant of a wild-type firefly luciferase comprising an amino acid sequence in which the amino acid residue at the position corresponding to position 393 of SEQ ID NO 1 is cysteine, wherein the luciferase mutant comprises an amino acid sequence in which the amino acid at the position is a non-acidic amino acid other than the cysteine.
(3) A luciferase mutant having improved thermostability wherein the luciferase mutant is a mutant of firefly luciferase, wherein the luciferase mutant comprises an amino acid sequence in which the amino acid at the position corresponding to position 393 of SEQ ID NO 1 is selected from the group consisting of leucine, proline, valine, isoleucine, histidine, methionine, alanine, phenylalanine, glutamine, tryptophan, tyrosine, serine, glycine, asparagine, lysine, threonine, and arginine.
(4) A luciferase mutant having improved thermostability, wherein the luciferase mutant is a mutant of a wild-type firefly luciferase comprising an amino acid sequence in which the amino acid residue at the position corresponding to position 393 of SEQ ID NO 1 is tyrosine, wherein the luciferase mutant comprises an amino acid sequence in which the amino acid at the position is an amino acid other than tyrosine or cysteine.
(5) The luciferase mutant according to any one of (1) to (4), wherein the amino acid at the position corresponding to position 393 of SEQ ID NO 1 is selected from the group consisting of leucine, proline, valine, isoleucine, histidine, methionine, and alanine.
(6) The luciferase mutant according to (5), wherein the amino acid at the position corresponding to position 393 of SEQ ID NO 1 is leucine, proline, or valine.
(7) The luciferase mutant according to any one of (1) to (6), wherein the amino acid at the position corresponding to position 217 of SEQ ID NO 1 is leucine or isoleucine, the amino acid at the position corresponding to position 490 of SEQ ID NO 1 is lysine, and/or the amino acid at the position corresponding to position 252 of SEQ ID NO 1 is methionine.
(8) The luciferase mutant according to any one of (1) to (7), wherein the luciferase mutant comprises an amino acid sequence selected from the group consisting of the following (i) to (iii):
(i) an amino acid sequence selected from the group consisting of SEQ ID NOs 1, 3, 5, and 7;
(ii) an amino acid sequence in which one or several amino acids are substituted, deleted, or added at a position other than the position corresponding to position 393 of SEQ ID NO 1 in any of the amino acid sequences in (i); and
(iii) an amino acid sequence having a sequence identity of 70% or more over the full length with any of the amino acid sequences in (i) and having a sequence identity of 90% or more between a region consisting of an amino acid sequence of the following positions of SEQ ID NO 1: positions 4 and 5, positions 9 and 10, positions 13 and 14, positions 16 and 17, position 19, position 23, positions 25 and 26, position 28, positions 35 to 37, position 40, positions 42 and 43, position 45, position 47, position 55, position 57, position 62, position 65, positions 72 to 74, position 80, positions 83 to 86, positions 90 and 91, position 93, position 98, position 101, positions 105 and 106, position 111, positions 114 to 116, position 119, position 122, position 125, position 129, positions 131 and 132, position 137, position 141, position 151, position 153, position 155, position 159, position 162, position 164, position 169, position 183, position 190, positions 195 and 196, position 198, positions 200 to 202, positions 204 and 205, positions 208 to 210, position 212, position 214, positions 220 to 223, positions 226 and 227, positions 230 and 231, position 235, positions 237 and 238, position 240, position 242, positions 244 to 251, positions 253 to 257, positions 260 to 263, position 270, position 272, positions 275 and 276, positions 279 to 283, position 286, positions 289 to 293, position 300, position 302, positions 305 to 309, position 311, positions 313 to 324, positions 326 and 327, position 329, positions 332 and 333, position 335, positions 339 to 350, positions 353 to 355, position 358, positions 361 and 362, positions 365 and 366, positions 368 and 369, positions 374 and 375, position 377, position 380, position 382, positions 384 and 385, position 387, positions 390 and 391, position 396, position 398, position 400, position 403, position 406, positions 408 to 410, position 414, positions 418 to 420, positions 423 to 425, position 427, position 429, positions 433 and 434, positions 436 to 451, positions 453 to 455, position 457, positions 460 to 464, position 466, positions 468 to 471, position 473, positions 475 and 476, positions 479 to 483, position 485, positions 487 and 488, positions 492 and 493, position 497, position 504, positions 506 to 508, positions 511 and 512, positions 514 to 518, position 520, positions 522 and 523, positions 525 to 527, positions 529 to 531, position 533, position 538, position 543, and position 547 and a region consisting of an amino acid sequence of positions corresponding to the positions in the luciferase mutant.
(9) The luciferase mutant according to (8), wherein the luciferase mutant comprises an amino acid sequence selected from the group consisting of the following (i) to (iii):
(i) the amino acid sequence of SEQ ID NO 1;
(ii) an amino acid sequence in which one or several amino acids are substituted, deleted, or added at positions other than the position corresponding to position 393 of SEQ ID NO 1 in the amino acid sequence in (i); and
(iii) an amino acid sequence having a sequence identity of 90% or more with the amino acid sequence in (i).
(10) A polynucleotide encoding the luciferase mutant according to any one of (1) to (9).
(11) A vector comprising the polynucleotide according to (10).
(12) A host cell comprising the polynucleotide according to (10) or the vector according to claim 11).
(13) A production method of a luciferase mutant having improved thermostability comprising a step of culturing the host cell according to (12).
(14) A kit for detecting at least one of ATP, ADP, or AMP comprising the luciferase mutant according to any one of (1) to (9).
(15) A method for detecting at least one of ATP, ADP, or AMP comprising using the luciferase mutant according to any one of (1) to (9).

The present specification encompasses the disclosed contents of Japanese Patent Application No. 2018-129384, which is the basis of the priority of the present application.

### Advantageous Effects of Invention

The present invention provides a firefly luciferase having improved thermostability.

### BRIEF DESCRIPTION OF DRAWINGS

FIG. 1-1 illustrates alignment results of wild-type luciferases of Luciola lateralis, Luciola cruciata, Photinus pyralis, and Photuris pennsylvanica. In the drawing, the same amino acid residues in four amino acid sequences are enclosed by frames.

FIG. 1-2 continues from FIG. 1-1.

### DESCRIPTION OF EMBODIMENTS

### (Luciferase Mutant)

In one aspect, the present invention relates to a mutant of firefly luciferase, for example, wild-type firefly luciferase, comprising an amino acid sequence in which the amino acid residue at the position corresponding to position 393 of SEQ ID NO 1 is substituted. In one aspect, the present invention relates to a mutant of firefly luciferase, for example, wild-type firefly luciferase, comprising an amino acid sequence in which the amino acid residue at the position corresponding to position 393 of SEQ ID NO 1 is cysteine. In one embodiment, thermostability of the luciferase mutant of the present invention is improved.

In the present specification, “wild-type” refers to a trait present the most in nature in a conspecific group.

Firefly luciferase derived from any firefly can be used as the firefly luciferase of the present invention. For example, Luciola lateralis, Luciola cruciata, Photinus pyralis, Photuris pennsylvanica, Lampyris noctiluca, Pyrocoelia miyako, Pyrophorus plagiophthalamus, or Luciola mingrelica, preferably the firefly luciferase from Luciola lateralis, Luciola cruciata, Photinus pyralis, or Photuris pennsylvanica can be used. Alternatively, chimeric proteins produced based on luciferase genes derived from various kinds of fireflies may be used.

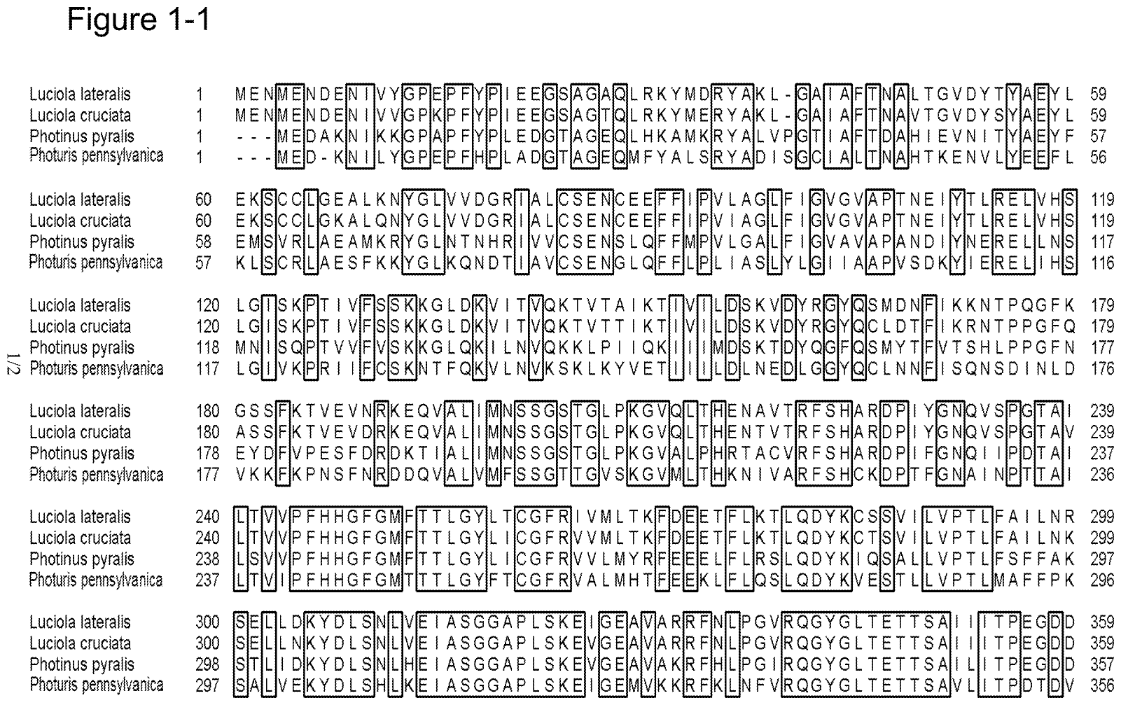

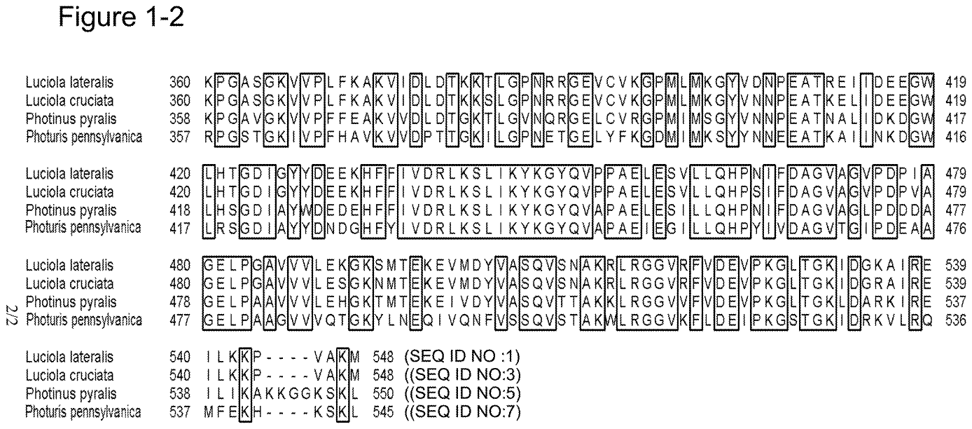

In the present specification, a correspondence relationship of amino acid positions can readily be identified through comparison of amino acid sequences of various kinds of firefly luciferases using, for example, the existing software for homology analysis of amino acids, for example, GENETYX (manufactured by GENETYX CORPORATION) or the like. For example, the amino acid position of a luciferase corresponding to position X in the amino acid sequence of SEQ ID NO 1 can be identified by aligning the amino acid sequence of the luciferase with the amino acid sequence of SEQ ID NO 1. For example, the “position corresponding to position 393 of SEQ ID NO 1” may be position 393 in the amino acid sequence of SEQ ID NO 3, position 391 of SEQ ID NO 5, and position 390 of SEQ ID NO 7. FIG. 1 illustrates alignment results of Luciola lateralis luciferase, Luciola cruciata luciferase, Photinus pyralis luciferase, and Photuris pennsylvanica luciferase. A corresponding position in each amino acid sequence can be determined with reference to such alignment results.

In one embodiment, the amino acid at the position corresponding to position 393 of SEQ ID NO 1 in the luciferase mutant is a non-acidic amino acid other than cysteine (that is, leucine, proline, valine, isoleucine, histidine, methionine, alanine, phenylalanine, glutamine, tryptophan, tyrosine, serine, glycine, asparagine, lysine, threonine, or arginine). In this embodiment, the luciferase mutant may be a luciferase mutant derived from Luciola lateralis, or a mutant comprising an amino acid sequence having a high sequence identity with the amino acid sequence of SEQ ID NO 1, for example, a sequence identity of 95% or more, 96% or more, 97% or more, 98% or more, or 99% or more. The amino acid at the position corresponding to position 393 of SEQ ID NO 1 is preferably selected from the group consisting of leucine, proline, valine, isoleucine, histidine, methionine, alanine, phenylalanine, glutamine, tryptophan, tyrosine, serine, and glycine, further preferably selected from the group consisting of leucine, proline, valine, isoleucine, histidine, methionine, and alanine, and more preferably is leucine, proline, or valine.

In one embodiment, the amino acid at the position corresponding to position 393 of SEQ ID NO 1 (for example, the amino acid at position 393 of SEQ ID NO 3) in a luciferase mutant is an amino acid other than cysteine. In this embodiment, the luciferase mutant may be a luciferase mutant derived from Luciola cruciata or a mutant comprising an amino acid sequence having a high sequence identity with the amino acid sequence of SEQ ID NO 3, for example, a sequence identity of 95% or more, 96% or more, 97% or more, 98% or more, or 99% or more. The amino acid at the position corresponding to position 393 of SEQ ID NO 1 may be asparagine, alanine, serine, arginine, leucine, threonine, histidine, valine, phenylalanine, glycine, tryptophan, tyrosine, isoleucine, proline, methionine, or glutamine, and, for example, may be asparagine, alanine, serine, arginine, leucine, threonine, histidine, or valine.

In one embodiment, the amino acid at the position corresponding to position 393 of SEQ ID NO 1 (for example, the amino acid at position 391 of SEQ ID NO 5) in a luciferase mutant is tryptophan. In this embodiment, the luciferase mutant may be a luciferase mutant derived from Photinus pyralis or a mutant comprising an amino acid sequence having a high sequence identity with the amino acid sequence of SEQ ID NO 5, for example, a sequence identity of 70% or more, 80% or more, 90% or more, 95% or more, 96% or more, 97% or more, 98% or more, or 99% or more.

In one aspect, the present invention relates to a luciferase mutant having improved thermostability comprising an amino acid sequence in which the amino acid at the position corresponding to position 393 of SEQ ID NO 1 is selected from the group consisting of leucine, proline, valine, isoleucine, histidine, methionine, alanine, phenylalanine, glutamine, tryptophan, tyrosine, serine, glycine, asparagine, lysine, threonine, and arginine. In this aspect, while the amino acid at the position corresponding to position 393 of SEQ ID NO 1 in a firefly luciferase, for example, the wild-type firefly luciferase, before introduction of the mutation is not limited as long as it is not any of the above-described amino acids, the amino acid may be, for example, tyrosine (with the proviso that, in this case, the amino acid after the substitution is an amino acid other than tyrosine). In one embodiment, the amino acid at the position corresponding to position 393 of SEQ ID NO 1 in a luciferase mutant is not tryptophan. In one embodiment, the amino acid at the position corresponding to position 393 of SEQ ID NO 1 in the luciferase mutant is preferably selected from the group consisting of leucine, proline, valine, isoleucine, histidine, methionine, and alanine, and more is preferably leucine, proline, or valine.

In one aspect, the present invention relates to a mutant of a firefly luciferase comprising an amino acid sequence in which the amino acid residue at the position corresponding to position 393 of SEQ ID NO 1 (for example, position 390 of SEQ ID NO 7) is tyrosine, wherein the luciferase mutant comprises an amino acid sequence in which the amino acid at said position is an amino acid other than tyrosine or cysteine (for example, an amino acid sequence comprising an amino acid other than tyrosine, cysteine, or tryptophan at said position) and has improved thermostability. In this aspect, the amino acid residue at the position corresponding to position 393 of SEQ ID NO 1 may be proline, asparagine, arginine, glycine, serine, lysine, phenylalanine, aspartic acid, glutamine, threonine, glutamic acid, isoleucine, or alanine, and may be, for example, proline, asparagine, arginine, glycine, serine, lysine, phenylalanine, aspartic acid, glutamine, or threonine.

In one embodiment, the mutation at the position corresponding to position 393 of SEQ ID NO 1 is artificially introduced. This can be achieved by artificially introducing the mutation in a sequence of a gene encoding luciferase.

In one embodiment, the firefly luciferase mutant of the present invention may further comprise a mutation other than the mutation at position 393 or the position corresponding to position 393 above. The mutation may be artificially introduced intending some specific effect or may be randomly or non-artificially introduced. Examples of mutations introduced with the purpose (intention) of obtaining a specific effect include addition or modification of the sequence to enhance the firefly luciferase gene expression level, a modification to improve purification efficiency of the firefly luciferase, and various kinds of mutations that give a practically preferred property to firefly luciferases. Examples of such publicly known mutations include mutations that enhances luminescence persistency as described in JP 2000-197484 A, mutations that change the emission wavelength as described in JP H03-285683 A (1991) or JP 2003-512071 T, mutations that enhance surfactant resistance as described in JP H-239493 A (1999), mutations that change substrate affinity as described in WO 99/02697, JP H10-512750 T (1998), or JP 2001-518799 T, mutations that enhance stability as described in JP H05-244942 A (1993), JP 2011-120559 A, JP 2000-197487 A, JP H09-510610 T (1997), or JP 2003-518912 T, mutations that improve luminescence persistency, stability, and luminescence amount as described in JP 201.1-188787 A, or the like. For example, JP 2011-120559 A discloses that thermostability is improved in firefly luciferase of Luciola lateralis luciferase having an amino acid sequence in which the amino acid equivalent to position 287 is mutated to alanine or the amino acid equivalent to position 392 is mutated to isoleucine. JP 2011-120559 A describes that combinations of these mutations, a mutation in which the amino acid at position 326 is substituted by serine, and/or a mutation in which the amino acid at position 467 is substituted with isoleucine allow obtaining firefly luciferases with further improved stability.

For example, in the luciferase mutant of the present invention, the amino acid at the position corresponding to position 217 of SEQ ID NO 1, may be leucine or isoleucine and/or the amino acid at the position corresponding to position 490 of SEQ ID NO 1 may be lysine. Additionally, in the luciferase mutant of the present invention, the amino acid at the position corresponding to position 252 of SEQ ID NO 1 may be methionine.

Examples of the firefly luciferase mutant of the present invention include: a mutant in which leucine is introduced at the position corresponding to position 217 of SEQ ID NO 1 and lysine is introduced at the position corresponding to position 490 (the amino acid sequence is indicated by SEQ ID NO 9) to wild-type Luciola lateralis luciferase (SEQ ID NO 1); a mutant in which isoleucine is introduced at the position corresponding to position 217 of SEQ ID NO 1 (position 217 of SEQ ID NO 3) to wild-type Luciola cruciata luciferase (SEQ ID NO 3); and a mutant in which methionine is introduced at the position corresponding to position 252 of SEQ ID NO 1 (position 249 of SEQ ID NO 7) to wild-type Photuris pennsylvanica luciferase (SEQ ID NO 7). These mutants may further comprising a mutation at position 393 or the position corresponding to position 393.

In one embodiment, the luciferase mutant comprises an amino acid sequence that comprises an amino acid mutation at the position corresponding to position 393 of SEQ ID NO 1 (and any other amino acid mutation described in the present specification) and selected from the group consisting of the following (i) to (iii);

(i) an amino acid sequence selected from the group consisting of SEQ ID NOs 1, 3, 5, and 7;

(ii) an amino acid sequence in which one or several amino acids are substituted, deleted, or added at a position other than the position corresponding to position 393 of SEQ ID NO 1 in any of the amino acid sequences in (i); and

(iii) an amino acid sequence having a sequence identity of 70% or more over the full length with any of the amino acid sequences in (i) and having a sequence identity of 90% or more between a region consisting of an amino acid sequence of the following positions of SEQ ID NO 1: positions 4 and 5, positions 9 and 10, positions 13 and 14, positions 16 and 17, position 19, position 23, positions 25 and 26, position 28, positions 35 to 37, position 40, positions 42 and 43, position 45, position 47, position 55, position 57, position 62, position 65, positions 72 to 74, position 80, positions 83 to 86, positions 90 and 91, position 93, position 98, position 101, positions 105 and 106, position 111, positions 114 to 116, position 119, position 122, position 125, position 129, positions 131 and 132, position 0.137, position 141, position 151, position 153, position 155, position 159, position 162, position 164, position 169, position 183, position 190, positions 195 and 196, position 198, positions 200 to 202, positions 204 and 205, positions 208 to 210, position 212, position 214, positions 220 to 223, positions 226 and 227, positions 230 and 231, position 235, positions 237 and 238, position 240, position 242, positions 244 to 251, positions 253 to 257, positions 260 to 263, position 270, position 272, positions 275 and 276, positions 279 to 283, position 286, positions 289 to 293, position 300, position 302, positions 305 to 309, position 311, positions 313 to 324, positions 326 and 327, position 329, positions 332 and 333, position 335, positions 339 to 350, positions 353 to 355, position 358, positions 361 and 362, positions 365 and 366, positions 368 and 369, positions 374 and 375, position 377, position 380, position 382, positions 384 and 385, position 387, positions 390 and 391, position 396, position 398, position 400, position 403, position 406, positions 408 to 410, position 414, positions 418 to 420, positions 423 to 425, position 427, position 429, positions 433 and 434, positions 436 to 451, positions 453 to 455, position 457, positions 460 to 464, position 466, positions 468 to 471, position 473, positions 475 and 476, positions 479 to 483, position 485, positions 487 and 488, positions 492 and 493, position 497, position 504, positions 506 to 508, positions 511 and 512, positions 514 to 518, position 520, positions 522 and 523, positions 525 to 527, positions 529 to 531, position 533, position 538, position 543, and position 547 (hereinafter also referred to as “identical region”) and a region consisting of an amino acid sequence of positions corresponding to these positions in the luciferase mutant.

In one embodiment, the amino acid sequence in (iii) has a sequence identity of 71% or more, 72% or more, 73% or more, 74% or more, 75% or more, 76% or more, 77% or more, 78% or more, 79% or more, preferably 80% or more, 81% or more, 82% or more. 83% or more, 84% or more, 85% or more, 86% or more, 87% or more, 88% or more, 89% or more, more preferably 90% or more, 91% or more, 92% or more, 93% or more, 94% or more, further preferably 95% or more, 96% or more, 97% or more, further more preferably 98% or more, and the most preferably 99% or more over the full length with any of the amino acid sequences in (i). Additionally, the “identical region” in the amino acid sequence in (iii) can be identified as regions where the same amino acid residues are preserved in the four firefly luciferases (Luciola lateralis, Luciola cruciata, Photinus pyralis, and Photuris pennsylvanica) illustrated in FIG. 1. In the amino acid sequence in (iii), the identical region and the region corresponding to the identical region in the luciferase mutant have a sequence identity of 91% or more, 92% or more, 93% or more, 94% or more, preferably 95% or more, 96% or more, 97% or more, more preferably 98% or more, and most preferably 99% or more.

In the present specification, the identity between an amino acid sequence and a gene sequence can be calculated by a program, such as maximum matching and search homology of GENETYX (manufactured by GENETYX CORPORATION), or a program, such as multiple alignment of CLUSTAL W and pairwise alignment by BLAST. When two or more luciferases are aligned for calculation of the amino acid sequence identity, positions of amino acids that are identical in these two or more luciferases can be examined. The identical areas in the amino acid sequences can be determined based on such information. In the present specification, with regard to two or more amino acid sequences, percent identity (%) refers to the percentage when the total number of amino acids in the alignable region is defined as the denominator and the number of positions occupied by the same amino acids among them is defined as the numerator in a case when the two or more amino acid sequences are aligned using BLAST (BLASTP) or the like on amino acid sequences. Therefore, when a region where no identity is observable is present in the two or more amino acid sequences, for example, when an additional sequence where no identity is observed is present at C-terminus in one amino acid sequence, the region is not used for the calculation of percent identity since the region with no identity cannot be aligned.

In the present specification, the range of “one or several” is from 1 to 10, preferably from 1 to 7, further preferably from 1 to 5, and particularly preferably from 1 to 3 or 1 or 2.

In one embodiment, the luciferase mutant comprises an amino acid mutation at the position corresponding to position 393 of SEQ. ID NO 1 (and any other amino acid mutation described in the present specification) and comprises an amino acid sequence selected from the group consisting of the following (i) to (iii);

(i) the amino acid sequence of SEQ ID NO 1;

(ii) an amino acid sequence in which one or several amino acids are substituted, deleted, or added at a position other than the position corresponding to position 393 of SEQ ID NO 1 in the amino acid sequence in (i); and

(iii) an amino acid sequence having a sequence identity of 90% or more, 91% or more, 92% or more, 93% or more, 94% or more, preferably 95% or more, 96% or more, 97% or more, more preferably 98% or more, and the most preferably 99% or more with the amino acid sequence in (i).

In one embodiment, the luciferase mutant of the present invention has luciferase activity. The presence or absence of the luciferase activity can be measured, for example, in accordance with the method described in Examples using Lumitester C-110 (manufactured by Kikkoman Biochemifa Company).

In the present specification, “thermostability” can be evaluated by using, for example, the residual activity when a heat treatment is performed on the firefly luciferase at a predetermined temperature for a predetermined period as an indicator. Specifically, the thermostability of the firefly luciferase in the present invention can be evaluated by comparing residual activity rates of the firefly luciferases after a heat treatment under a high temperature condition, for example, at a reaction temperature usually from 30 to 50° C., for example, from 35 to 45° C. or from 35 to 40° C. for a certain period, usually from 5 to 180 minutes or from 10 to 180 minutes, for example, from 60 to 180 minutes or about 90 minutes. The residual activity rate of the firefly luciferase of the present invention is calculated as a ratio of the firefly luciferase activity after the heat treatment to the firefly luciferase activity before the action under the above-described high temperature condition. Improved thermostability in the present invention refers to a case where the residual activity rate when the firefly luciferase mutant is applied under the above-described conditions is improved by 1.01 times or more, 1.02 times or more, 1.1 times or more, 1.2 times or more, and preferably 1.4 times or more or 1.5 times or more relative to the luciferase to which the mutation of the present invention (the mutation at the position corresponding to position 393 of SEQ ID NO 1) is not introduced, for example, the wild-type luciferase or the luciferase comprising the amino acid sequences that are the same as the amino acid sequences other than the mutated amino acid sequence of the present invention.

### (Polynucleotide)

In one aspect, the present invention relates to a polynucleotide (hereinafter also referred to as “luciferase gene”) encoding the luciferase mutant of the present invention. A sequence of the polynucleotides can readily be determined based on the amino acid sequence of the firefly luciferase mutant. For example, the polynucleotides encoding the amino acid sequences of SEQ ID NOs 1, 3, 5, and 7 are polynucleotides of SEQ ID NOs 2, 4, 6, and 8, respectively. For example, the polynucleotide of the present invention may comprise:

(i) a nucleotide sequence selected from the group consisting of SEQ ID NOs 2, 4, 6, and 8;

(ii) a nucleotide sequence in which one or several nucleotides are substituted, deleted, or added in any of the nucleotide sequences in (i); and

(iii) a nucleotide sequence having a sequence identity of 70% or more, 71% or more, 72% or more, 73% or more, 74% or more, 75% or more, 76% or more, 77% or more, 78% or more, 79% or more, preferably 80% or more, 81% or more, 82% or more, 83% or more, 84% or more, 85% or more, 86% or more, 87% or more, 88% or more, 89% or more, more preferably 90% or more, 91% or more, 92% or more, 93% or more, 94% or more, further preferably 95% or more, 96% or more, 97% or more, further more preferably 98% or more, and the most preferably 99% or more with any of the nucleotide sequences in (i) over the full length.

In order to obtain nucleotides encoding these luciferases, methods for cloning genes used in general can be applied. For example, chromosomal DNA or mRNA can be extracted from tissues or cells of a firefly having a luciferase producing ability by conventional methods, for example, the method described in Current Protocols in Molecular Biology (WILEY Interscience, 1989). Further, cDNA can be synthesized using the mRNA as the template. The chromosomal DNA or cDNA thus obtained can be used to produce a library of the chromosomal DNA or the cDNA.

Next, by using a method in which an appropriate probe DNA is synthesized based on the amino acid sequence of the luciferase and using the same and selecting the polynucleotide encoding the luciferase from the library of the chromosomal DNA or cDNA, or by producing an appropriate primer DNA based on the amino acid sequence, amplifying DNA comprising the objective nucleotide fragment encoding the luciferase with an appropriate Polymerase Chain Reaction (PCR method), such as a 5′RACE method or a 3′RACE method, and ligating these DNA fragments, it is possible to obtain DNA comprising the full length nucleotide encoding the objective luciferase.

Further, when the nucleotide sequence encoding the luciferase is already-known, as in, for example, nucleotide sequences shown in SEQ ID NOs 2, 4, 6, and 8, the nucleotide sequence may be artificially synthesized. Artificial gene synthesizing service is provided by, for example, Integrated DNA Technologies.

### (Production Method of Luciferase Gene)

The mutation treatment of the luciferase gene can be performed by any known method according to the intended mutation form. That is, a method that brings the luciferase gene or recombinant DNA, into which this gene is incorporated, into contact with an agent serving as a mutagen so as to allow the agent to act on the gene or DNA; an ultraviolet irradiation method; a genetic engineering method; a method making full use of protein engineering methods, or the like can be widely used.

Examples of the agent serving as the mutagen used for the mutation treatment above include hydroxylamine, N-methyl-N′-nitro-N-nitrosoguanidine, nitrous acid, sulfurous acid, hydrazine, formic acid, 5-bromouracil, and the like.

Regarding various conditions of contacting and allowing to act, conditions depending on the type of agent being used or the like can be employed, and the conditions are not specifically limited as long as a desired mutation can be actually induced in the luciferase gene. Usually, the contact and action with the agent at a concentration of preferably 0.5 to 12 M, a reaction temperature of 20 to 80° C. for 10 minutes or more and preferably from 10 to 180 minutes allows for inducing the desired mutation. When ultraviolet irradiation is performed, it can be carried out in accordance with conventional methods as described above (Modern Chemistry, 024 to 30, June 1.989 issue).

As a method making full use of protein engineering methods, in general, a method known as Site-Specific Mutagenesis can be used. Examples include the Kramer method (Nucleic Acids Res., 12, 9441 (1984): Methods Enzymol., 154, 350 (1987): Gene, 37, 73 (1985)), the Eckstein method (Nucleic Acids Res., 13, 8749 (1985): Nucleic Acids Res., 13, 8765 (1985): Nucleic Acids Res, 14, 9679 (1986)), the Kunkel method (Proc. Natl. Acid. Sci. U.S.A., 82, 488 (1985): Methods Enzymol., 154, 367 (1987)), and the like. Additionally, Site-Specific Mutagenesis can be performed using a commercially available kit, for example, QuickChange Site-Directed Mutagenesis Kit (manufactured by Agilent Technologies).

Alternatively, a method generally known as a PCR method can also be used (see Technique, 1, 11 (1989)). Incidentally, apart from the genetic modification method above, a desired modified luciferase gene can be directly synthesized using an organic synthesis method or an enzyme synthesis method.

When determining or confirming the DNA nucleotide sequence of the luciferase gene obtained by the method above, for example, a multi-capillary DNA analysis system, Applied Biosystems 3130xl genetic analyzer (manufactured by Thermo Fisher Scientific) or the like can be used.

### (Vector, Host Cell)

In one aspect, the present invention relates to a vector comprising the polynucleotide. It is preferable that these luciferase genes are ligated to various types of vectors in accordance with conventional method for ease of handling. A plasmid can be used as the vector of the present invention, and apart from this, any vector known to those skilled in the art, such as a bacteriophage, a cosmid, and the like can be used. The type of the vector can be selected depending on the host cell, and specifically, for example, pET16-b, pKK223-3, or the like is preferred.

In one aspect, the present invention relates to a host cell comprising the polynucleotide or vector. Although not limited, the host cell is a bacteria, such as Escherichia coli and Bacillus subtilis, a yeast cell, an insect cell, an animal cell (for example, a mammal cells) a plant cell, or the like, and preferably a bacterial cell, such as Escherichia coli and the like.

### (Transformation and Transduction)

The luciferase gene obtained as described above can be incorporated into a vector, such as a bacteriophage, cosmid, or plasmid used for transformation of a prokaryotic cell or an eukaryotic cell, by conventional methods, and the transformation or transduction can be performed on the host corresponding to each vector by conventional methods. For example, using a microorganism belonging to the genus Escherichia as the host, for example, and by using the obtained recombinant DNA, for example, Escherichia coli K-12 strain or Escherichia coli B strain, preferably Escherichia coli JM109 strain, Escherichia coli DH5α strain, Escherichia coli BL21 strain, Escherichia coli BL21 (DE3) strain (all of them are manufactured by Takara Bio), and the like can be transformed or transduced to obtain the respective strain.

### (Production Method of a Luciferase Mutant)

In one aspect, the present invention relates to the production method of the luciferase mutant having improved thermostability comprising a step of culturing the host cell. Culturing can be performed by various kinds of known methods, and a solid culture method may be used, but is preferably performed by a liquid culture method.

The method of the present invention may comprise a step of culturing the host cell under a condition under which a luciferase protein can be expressed and optionally a step of isolating the luciferase from the culture product or culture fluid. Here, the condition under which the luciferase protein can be expressed refers to conditions under which transcription and translation of a luciferase gene and production of the polypeptide encoded by this gene takes place.

In one embodiment, the method of the present invention comprises artificially introducing a mutation at the position corresponding to position 393 of SEQ ID NO 1 in a luciferase protein before the culturing step. This can be performed by artificially introducing the mutation to the sequence of a gene encoding the luciferase.

As the medium for culturing the host cell, for example, one produced by adding one or more kinds of inorganic salts, such as sodium chloride, potassium di hydrogen phosphate, dipotassium hydrogenphosphate, magnesium sulfate, magnesium chloride, ferric chloride, ferric sulfate, or manganese sulfate to one or more kinds of nitrogen sources, such as a yeast extract, triptone, peptone, a meat extract, corn steep liquor, or exudate of soybean or wheat bran and further adding a carbohydrate raw material, vitamin, or the like as necessary is used.

The initial pH of the medium is appropriately adjusted to pH 7 to 9. Culturing is performed at a cultivation temperature of 20° C. to 42° C., preferably at a cultivation temperature of around 25° C. to 37° C. for 4 to 24 hours, and further preferably at a cultivation temperature of around 25° C. to 37° C. for 8 to 16 hours preferably by aeration-agitation submerged culture, shake culture, static culture, or the like.

After completion of culturing, and in order to collect the luciferase from the cultured product, conventional means for collecting enzymes can be employed. For example, the present enzyme can be released from a microorganism body by performing ultrasonic disruption treatment, milling treatment, or the like on the microorganism body by conventional methods, extracting the present enzyme with lytic enzymes, such as lysozyme, or shaking or allowing to stand till in the presence of toluene or the like for lysis. This solution is, for example, filtrated or centrifuged to remove solid matter, and nucleic acids are removed with streptomycin sulfate, protamine sulfate, manganese sulfate, or the like if necessary and subsequently, ammonium sulfate, alcohol, acetone, or the like is added thereto and fractionation is performed, a precipitate is collected, and a crude enzyme of the luciferase is obtained.

In order to further obtain a purified luciferase enzyme from the crude enzyme of the luciferase, for example, a gel filtration method using Sephadex, Superdex, Ultro-gel, or the like; an adsorption elution method using an ion exchange carrier, a hydrophobic carrier, or hydroxyapatite; an electrophoresis method using polyacrylamide gel or the like; a sedimentation method, such as a sucrose density-gradient centrifugation; an affinity chromatography method; or a fractionation method using a molecular sieving membrane, a hollow fiber membrane, or the like is appropriately selected and performed, or a combination of the same is performed, to obtain the purified luciferase enzyme. The desired luciferase can be thus obtained.

The luciferase produced by the method of the present invention can be used in a kit described in the present specification or by a method for detecting at least one of ATP, ADP, or AMP.

### (Kit for Detecting at Least One of ATP, ADP, or AMP)

In one aspect, the present invention relates to the kit comprising the luciferase mutant described in the present specification for detecting at least one of ATP, ADP, or AMP. The kit of the present invention may comprise luciferin in addition to the luciferase mutant. In this case, metal ions, such as magnesium, manganese, and calcium, can also be included in the kit. Those skilled in the art can determine the concentration of the metal ions depending on the enzyme being used. Luciferase converts ATP, 07, and the luciferin into AMP, pyrophosphoric acid, CO₂, and oxyluciferin, and during this conversion luminescence is provided. The reaction that occurs during this conversion is expressed as follows.

Luciferin+ATP+O₂→oxyluciferin+adenosine monophosphate (AMP) pyrophosphoric acid (PPi)+CO₂+light

In one embodiment, the kit of the present invention further comprises an enzyme catalyzing a reaction of generating ATP from ADP. This enzyme catalyzing the reaction of generating ATP from ADP can be selected from the group consisting of pyruvate kinase (PK), acetate kinase (AK), creatine kinase (CK), polyphosphate kinase (PPK), hexokinase, glucokinase, glycerol kinase, fructokinase, phosphofructokinase, riboflavin kinase, and fructose-bisphosphatase. In another embodiment, the kit of the present invention further comprises pyruvate orthophosphate dikinase (PPDK), adenylate kinase (ADK), or pyruvate-water dikinase (PWDK).

If ATP is contained in a sample, it is converted into AMP by the luciferase and luminescence is also generated. If ADP is contained in a sample in a system where an enzyme catalyzing the reaction of generating ATP from ADP is present, said enzyme converts ADP into ATP and subsequently, ATP is subjected to a luminescence reaction. Due to the foregoing, the total quantity of ATP and ADP present in the system can be measured. Further, in a system where PPDK is present, if AMP is contained in a sample, this is converted into ATP by PPDK, PEP, and PPI. Alternatively, if AMP is contained in a sample in a system where PWDK is present, AMP is converted into ATP by PWDK, PEP, and phosphoric acid. The generated ATP produces luminescence again by the luciferase. Since the luminescence is stably maintained and since the amount of luminescence correlates with the total quantity of ATP and AMP present in the system, ATP and AMP can be quantitated. The presence of an enzyme catalyzing the reaction generating ATP from ADP and PPDK, ADK, or PWDK allows for measuring the total quantity of ATP, ADP, and AMP.

The luciferin may be any luciferin as long as it is recognized as a substrate by the luciferase being used and may be natural or chemically synthesized. Moreover, any known luciferin derivative can also be used. The basic structure of luciferin is imidazopyrazinone, and there are many tautomers thereof. Luciferin includes firefly luciferin. The firefly luciferin is a substrate of firefly luciferase (EC 1.13.12.7). The luciferin derivative can be those described in JP 2007-91695 A and JP 2010-523149 T (WO 2008/127677) and the like.

In addition to the components above, the kit of the present invention may include at least one of a stabilizing agent, a buffer, or instructions for use.

### (Method for Detecting at Least One of ATP, ADP, or AMP)

In one aspect, the present invention relates to a method that detects at least one of ATP, ADP, or AMP, comprising using the luciferase mutant described in the present specification. This method may comprise a step of catalyzing an oxidation reaction of the luciferin using the luciferase mutant described in the present specification and a step of measuring luminescence generated by the oxidation reaction.

The catalyst of the oxidation reaction of the luciferin by the luciferase mutant is as described in “Kit for detecting at Least one of ATP, ADP, or AMP.” This can be performed by causing the luciferase mutant and the luciferin described in the present specification to react with a sample. If ATP is contained in the sample, ATP is converted into AMP by the luciferase and luminescence is generated, and therefore ATP can be measured. In a system where an enzyme catalyzing the reaction generating ATP from ADP is present, the total quantity of ATP and ADP present in the system can be measured. Further, in a system where PPDK or PWDK is present, ATP and AMP can be quantitated. If an enzyme catalyzing the reaction generating ATP from ADP and PPDK, ADK, or PWDK is present, the total quantity of ATP, ADP, and AMP can be measured.

The amount of luminescence from the luciferase can be measured by known methods and can be evaluated using relative luminescence intensity (RLU) as the indicator which is obtained using, for example, an appropriate apparatus for measuring luminescence, for example, a luminometer (CentroLB960 or Lumat3 LB9508 manufactured by Berthold Technologies GmbH & Co. KG; Lumitester C-110, Lumitester C-100, Lumitester PD-20, or Lumitester PD-30 manufactured by Kikkoman Biochemifa Company, or the like). Typically, luminescence generated during conversion of luciferin to oxyluciferin is measured. As the apparatuses for measuring luminescence, apparatuses capable of high sensitivity measurement and equipped with a photomultiplier tube (those manufactured by 3M Corporation and the like) and apparatuses equipped with a photodiode (those manufactured by Hygiena, LLC, Neogen Corporation, and the like) can also be used.

The present invention will be described more specifically referring to the Examples below. However, the technical scope of the present invention is not limited by the Examples in any way.

### EXAMPLES

### Example 1: Thermal Resistance Test of a Mutant of Luciola Lateralis Luciferase

### (Materials and Methods)

### Vector Construction

To the multicloning site (MCS, Nde1-Bam1 site) of pET16-b (Novagen), the gene sequence of a construct with mutations A217L, and E490K introduced into HLK (wild-type Luciola lateralis luciferase (SEQ ID NO 1) to improve thermostability (amino acid sequence: SEQ ID NO 9, nucleotide sequence: SEQ ID NO 10) was inserted and the resulting plasmid pET-16b) was used as the template, and by carrying out PCR using primers for amplifying the sequences encoding each mutant, plasmid vectors encoding each respective mutant in which cysteine at position C393 was substituted with various types of amino acids were produced.

The sequence of the reverse primers used to produce the respective plasmid vectors is a common sequence which is SEQ ID NO I1 (AACTTCTCCACGTCTGTTCGGGCCCAAAG). The following table shows sequences and SEQ ID NOs of respective forward primers used to produce the plasmid vectors encoding luciferases comprising each respective amino acid at position 393.

TABLE 1

| Amino | | |
|---|---|---|
| acid at | | SEQ |
| position | | ID |
| 393 | Forward primer sequence | NO |
| Alanine | AGACGTGGAGAAGTTGCGGTAAAGGGTCC | 12 |
| Aspartic | AGACGTGGAGAAGTTGATGTAAAGGGTCC | 13 |
| acid | | |
| Glutamic | AGACGTGGAGAAGTTGAAGTAAAGGGTCC | 14 |
| acid | | |
| Phenyl- | AGACGTGGAGAAGTTTTTGTAAAGGGTCC | 15 |
| alanine | | |
| Glycine | AGACGTGGAGAAGTTGGCGTAAAGGGTCC | 16 |
| Histidine | AGACGTGGAGAAGTTCATGTAAAGGGTCC | 17 |
| Isoleucine | AGACGTGGAGAAGTTATTGTAAAGGGTCC | 18 |
| Lysine | AGACGTGGAGAAGTTAAAGTAAAGGGTCC | 19 |
| Leucine | AGACGTGGAGAAGTTCTGGTAAAGGGTCC | 20 |
| Methionine | AGACGTGGAGAAGTTATGGTAAAGGGTCC | 21 |
| Asparagine | AGACGTGGAGAAGTTAATGTAAAGGGTCC | 22 |
| Proline | AGACGTGGAGAAGTTCCGGTAAAGGGTCC | 23 |
| Glutamine | AGACGTGGAGAAGTTCAGGTAAAGGGTCC | 24 |
| Arginine | AGACGTGGAGAAGTTCGCGTAAAGGGTCC | 25 |
| Serine | AGACGTGGAGAAGTTAGCGTAAAGGGTCC | 26 |
| Threonine | AGACGTGGAGAAGTTACCGTAAAGGGTCC | 27 |
| Valine | AGACGTGGAGAAGTTGTGGTAAAGGGTCC | 28 |
| Tryptophan | AGACGTGGAGAAGTTTGGGTAAAGGGTCC | 29 |
| Tyrosine | AGACGTGGAGAAGTTTATGTAAAGGGTCC | 30 |

Subsequently, 10×KOD plus buffer (TOYOBO): 2.0 μlmM dNTPs: 2.0 μl, 25 mM MgSO₄: 1.2 μl, 2μM primer Fw: 3.0 μl, 2 μM primer Rv: 3.0 μl, KOD-plus-Neo (TOYOBO): 0.4 μl, 40 μg/ml pET16-b: 0.5 and dH₂O: 7.9 μl were mixed to produce a solution with a total of 20 μl and this was subjected to the PCR reaction. The PCR reaction was performed by heating at 94° C. for 2 minutes and then repeating a cycle of 94° C. for 15 seconds, 55° C. for 30 seconds, and 68° C. for 3 minutes 14 times, and after the reaction, the resultant was left to stand at 15° C.

### Transformation

1 μl of restriction enzyme DpnI (NewEngland Biolabs Japan) was added to the PCR product obtained above, the resultant was incubated at 37° C. for one hour, and the reaction product was used for transformation.

Competent cells (EGOS™ competent E. coli BL21 (DE3)) (NIPPON GENE CO., LTD.) were melted on an ice by 30 μl, and 3 μl of the reaction product was added immediately to the same. After tapping, the resultant was placed on the ice for 5 minutes and subsequently warmed at 42° C. for 30 seconds. After the warming, tapping was performed, and the entire amount was spread on a Luria-Bertani (LB)+ampicillin (Amp) plate and cultured overnight at 37° C., thus forming a colony.

### Preparation of Heat Resistant Mutant

Shake culturing (reciprocation) was performed overnight on the colony obtained above in 2 ml of an LB medium to which Amp was added such that the final concentration was 50 μg/ml. Subsequently, 2 μl of the cultured fluid was added to 2 ml of the LB medium to which AMP was added such that the final concentration was 50 μg/ml. Shake culturing (reciprocation) was performed at 28° C. for 22 hours, and isopropyl-β-thiogalactopyranoside (IPTG) was added such that the final concentration was 0.1 mM at the start of shaking, thus carrying out expression induction.

After collecting the bacteria, the resultant was suspended in 1 ml of a luciferase buffer (5% trehalose, 10 mM Tris, 4.4 mM succinic acid, 1 mM EDTA, and 1 mM DTT (pH 7.6)). The microorganism body was crushed using a sonicator, astrason ULTRASONIC PROCESSOR XL (manufactured by Misonix), (10 s pulse, 20 s rest, total pulse 1 min). The supernatant obtained by centrifugation was filtered with a 0.45 μm or 0.20 μm PVDF membrane to produce a crude enzyme liquid.

As the control for evaluation, the HLK into which the A217L and E490K mutations were introduced (SEQ ID NO 9) was expressed in accordance with the method described in JP H08-98680 A (1996), and a preliminarily purified enzyme was used.

### Heat Stabilization Test

Before storing the prepared enzyme at 37° C., the enzyme was pre-incubated at 25° C. for 5 minutes to return it to a room temperature from refrigeration. Subsequently, using a water bath, the enzyme was stored at 37° C. for 90 minutes and diluted as necessary using a dilution buffer (4.48 g of tricine, 185 mg of EDTA.Na₂.2H₂O, 25 g of glycerol, and 5 g of BSA (pH 7.8) per 500 ml) to prepare the enzyme so as to fall within the measurement range of Lumitester C-110. The same amounts of 100 μl of the prepared crude enzyme liquid or 100 μl of the HLK already purified and 100 μl of the luminescence reagent described below were mixed, and the amount of luminescence was measured using Lumitester C-110 (manufactured by Kikkoman Biochemifa Company).

As the luminescence reagent, a solution produced by mixing 2.0 ml of 50 mM tricine-NaOH buffer (pH 7.8), 0.5 ml of 40 mM ATP solution, 2.0 ml of 5.0 mM luciferin, and 0.5 ml of 0.1 M MgSO₄ was used.

The measuring conditions are as follows.

Timing of initiation of measurement: 10 seconds after addition of the luminescence reagent to the enzyme liquid
Measurement duration: integration of 10 seconds

### (Results)

The test was conducted three times on each luciferase to obtain an average value of the measured values. The following table shows relative values at 37° C. for 90 minutes storage period relative to when the storage period of 0 minutes at 37° C. is defined as 1 and also shows residual activity ratios of the respective mutants relative to the residual activity after 90 minutes without mutation defined as 1 (residual activity ratio after 90 minutes) (Table 2).

TABLE 2

| Mutation at | Storage period at 37° C. | | Residual activity ratio |
|---|---|---|---|
| position 393 | 0 minutes | 90 minutes | after 90 minutes |
| None | 1 | 0.49 | 1.00 |
| E | 1 | 0.19 | 0.39 |
| D | 1 | 0.27 | 0.55 |
| R | 1 | 0.50 | 1.02 |
| T | 1 | 0.51 | 1.04 |
| K | 1 | 0.53 | 1.08 |
| N | 1 | 0.57 | 1.17 |
| G | 1 | 0.62 | 1.25 |
| S | 1 | 0.63 | 1.29 |
| Y | 1 | 0.63 | 1.29 |
| W | 1 | 0.64 | 1.29 |
| Q | 1 | 0.65 | 1.32 |
| F | 1 | 0.67 | 1.35 |
| A | 1 | 0.70 | 1.43 |
| M | 1 | 0.74 | 1.50 |
| H | 1 | 0.76 | 1.54 |
| I | 1 | 0.79 | 1.60 |
| V | 1 | 0.80 | 1.63 |
| P | 1 | 0.81 | 1.64 |
| L | 1 | 0.81 | 1.65 |

As shown in Table 2, when C393 was substituted with an amino acid other than an acidic amino acid (E, D), improved thermal resistance was observed, and, in particular, when substituted with G (glycine), S (serine), Y (tyrosine), W (tryptophan), Q (glutamine), F (phenylalanine), A (alanine), M (methionine), (histidine), T (isoleucine), V (valine), P (proline), or L (leucine), the effect of improved thermal resistance was significant.

In the test system, the HLK used as the control is a purified enzyme not having a His tag, and the C393 mutants are crudely purified enzymes comprising 10 His tags at N-terminus in the sequence. Therefore, in order to confirm that the thermostability of the enzyme does not change depending on presence or absence of the His tag and purification, a purified enzyme of the HLK and a purified or a crudely purified HLK comprising 10 His tags at the N-terminus were prepared by methods identical to those described above, and heat resistance was tested by warming for 30 minutes or 60 minutes at 42° C. As a result, since the three of the purified HLK enzyme, purified HLK enzyme comprising the His tag, and crudely purified HLK enzyme comprising the His tag did not exhibit a difference in stability, it was considered that the presence or absence of the His tag and the presence or absence of purification did not affect the thermostability of the enzyme (data not shown).

### Example 2: Thermal Resistance Test of Mutants of Other Firefly Luciferases

### (Materials and Methods)

A gene sequence of wild-type Photinus pyralis luciferase (amino acid sequence: SEQ ID NO 5, nucleotide sequence: SEQ ID NO 6) was introduced into an MCS site of pET16-b (Nde1-BamH1 site). The obtained plasmid was termed Ppy pET16-b.

Further, gene sequences of wild-type Luciola cruciata luciferase (amino acid sequence: SEQ ID NO 3, nucleotide sequence: SEQ ID NO 4) into which a T217I mutation was introduced and wild-type Photuris pennsylvanica luciferase (amino acid sequence: SEQ ID NO 7, nucleotide sequence: SEQ ID NO 8) into which T249M mutation was introduced were introduced into MCS sites (EcoR1-HindIII sites) of pKK223-3. The obtained plasmids were termed LucT pKK223-3 and PpeT249M pKK223-3, respectively. Incidentally, as a gene encoding Luciola cruciata luciferase, a nucleotide sequence in which a mutation was introduced into a nucleotide sequence of SEQ ID NO 4 to include the T217I mutation and a nucleotide sequence of SEQ ID NO 74 in which a sequence encoding the His tag (6 Histidine residues) was added immediately before the stop codon was used. As a gene encoding Photuris pennsylvanica luciferase, codon optimization was performed on the nucleotide sequence of SEQ ID NO 8 and a mutation was introduced to include the T249M mutation, and further, a sequence encoding a His tag (6 Histidine residues) was added immediately before the stop codon to produce the nucleotide sequence of SEQ ID NO 31 which was used.

Using LucT pKK223-3, PpeT249M pKK223-3, and Ppy pET16-bg, plasmid vectors encoding the respective mutants produced by substitution of position C393 of Luciola cruciata luciferase, position Y390 of Photuris pennsylvanica luciferase, and position C391 of Photinus pyralis luciferase by various types of amino acids were produced in accordance with Example 1 using the following primers.

The sequence of the reverse primer used to produce plasmid vectors encoding the luciferases comprising each amino acid at position 393 of Luciola cruciata luciferases is a common sequence (SEQ ID NO 32 (AACTTCTCCACGTCTGTTAGGACCTAAAG)), and the following table shows sequences and SEQ ID NOs of the respective forward primers.

TABLE 3

| Amino | | |
|---|---|---|
| acid at | | SEQ |
| position | | ID |
| 393 | Forward primer sequence | NO |
| Alanine | AGACGTGGAGAAGTTGCGGTTAAAGGACC | 33 |
| Aspartic | AGACGTGGAGAAGTTGATGTTAAAGGACC | 34 |
| acid | | |
| Glutamic | AGACGTGGAGAAGTTGAAGTTAAAGGACC | 35 |
| acid | | |
| Phenyl- | AGACGTGGAGAAGTTTTTGTTAAAGGACC | 36 |
| alanine | | |
| Glycine | AGACGTGGAGAAGTTGGCGTTAAAGGACC | 37 |
| Histidine | AGACGTGGAGAAGTTCATGTTAAAGGACC | 38 |
| Isoleucine | AGACGTGGAGAAGTTATTGTTAAAGGACC | 39 |
| Lysine | AGACGTGGAGAAGTTAAAGTTAAAGGACC | 40 |
| Leucine | AGACGTGGAGAAGTTCTGGTTAAAGGACC | 41 |
| Methionine | AGACGTGGAGAAGTTATGGTTAAAGGACC | 42 |
| Asparagine | AGACGTGGAGAAGTTAATGTTAAAGGACC | 43 |
| Proline | AGACGTGGAGAAGTTCCGGTTAAAGGACC | 44 |
| Glutamine | AGACGTGGAGAAGTTCAGGTTAAAGGACC | 45 |
| Arginine | AGACGTGGAGAAGTTCGCGTTAAAGGACC | 46 |
| Serine | AGACGTGGAGAAGTTAGCGTTAAAGGACC | 47 |
| Threonine | AGACGTGGAGAAGTTACCGTTAAAGGACC | 48 |
| Valine | AGACGTGGAGAAGTTGTGGTTAAAGGACC | 49 |
| Tryptophan | AGACGTGGAGAAGTTTGGGTTAAAGGACC | 50 |
| Tyrosine | AGACGTGGAGAAGTTTATGTTAAAGGACC | 51 |

The sequence of the reverse primer used to produce plasmid vectors encoding the luciferases comprising each amino acid at position 390 of Photuris pennsylvanica luciferases is a common sequence (SEQ ID NO 52 (CAGTTCACCGGTTTCGTTCGGGCCCAGG)) except when aspartic acids is introduced to position 390. The following Table shows sequences and SEQ ID NOs of the respective forward primers and the reverse primer when aspartic acid is introduced to position 390.

TABLE 4

| Amino | | |
|---|---|---|
| acid at | | SEQ |
| position | | ID |
| 390 | Primer sequence | NO |
| Alanine | GAAACCGGTGAACTGGCGTTTAAAGGCG | 53 |
| Glutamic | GAAACCGGTGAACTGGAATTTAAAGGCG | 54 |
| acid | | |
| Phenyl- | GAAACCGGTGAACTGTTTTTTAAAGGCG | 55 |
| alanine | | |
| Glycine | GAAACCGGTGAACTGGGCTTTAAAGGCG | 56 |
| Histidine | GAAACCGGTGAACTGCATTTTAAAGGCG | 57 |
| Isoleucine | GAAACCGGTGAACTGATTTTTAAAGGCG | 58 |
| Lysine | GAAACCGGTGAACTGAAATTTAAAGGCG | 59 |
| Leucine | GAAACCGGTGAACTGCTGTTTAAAGGCG | 60 |
| Methionine | GAAACCGGTGAACTGATGTTTAAAGGCG | 61 |
| Asparagine | GAAACCGGTGAACTGAATTTTAAAGGCG | 62 |
| Proline | GAAACCGGTGAACTGCCGTTTAAAGGCG | 63 |
| Glutamine | GAAACCGGTGAACTGCAGTTTAAAGGCG | 64 |
| Arginine | GAAACCGGTGAACTGCGCTTTAAAGGCG | 65 |
| Serine | GAAACCGGTGAACTGAGCTTTAAAGGCG | 66 |
| Threonine | GAAACCGGTGAACTGACCTTTAAAGGCG | 67 |
| Valine | GAAACCGGTGAACTGGTGTTTAAAGGCG | 68 |
| Cysteine | GAAACCGGTGAACTGTGCTTTAAAGGCG | 69 |
| Aspartic | GAAACCGGTGAACTGGATTTTAAAGGCG | 70 |
| acid | (Forward) | |
| Aspartic | CGCCTTTAAAATCCAGTTCACCGGTTTC | 71 |
| acid | (Reverse) | |

The sequence of the forward primer used to produce the plasmid vector into which tryptophan is introduced at position 391 of Photinus pyralis luciferase is SEQ ID NO 72 (CAGAGAGGCGAATTATGGGTCAGAGGACC), and the sequence of the reverse primer is SEQ ID NO 73 (TAATTCGCCTCTCTGATTAACGCCCAGCG).

Details of vector construction, transformation, and preparation of heat resistant mutants were carried out as in Example 1.

While the heat stabilization test was also carried out as in Example 1, the following modifications were made. The warming period with the water bath was 90 minutes for Luciola cruciata luciferase, 5 minutes for Photuris pennsylvanica luciferase, and 20 minutes for Photinus pyralis luciferase. Additionally, as a control, a luciferase (LucT) produced by introducing T217I mutation into the wild-type Luciola cruciata luciferase for Luciola cruciata luciferase, a luciferase (PpeT249M) produced by introducing T249M mutation into the wild-type Photuris pennsylvanica luciferase for Photuris pennsylvanica luciferase, and the wild-type Photinus pyralis luciferase (Ppy) was used for Photinus pyralis luciferase. The preparation method of these controls is similar to the method described above. Incidentally, both of T217I and T249M are mutations known to improve heat resistance.

### (Results)

The test was conducted three times on Luciola cruciata luciferase to obtain an average value of the measured values. The following table shows relative values at 37° C. for 90 minutes storage period relative to when the storage period of 0 minutes at 37° C. is defined as 1 and also shows residual activity ratios of the respective mutants relative to the residual activity after 90 minutes without mutation defined as 1 (residual activity ratio after 90 minutes).

TABLE 5

| Mutation at | Storage period at 37° C. | | Residual activity ratio |
|---|---|---|---|
| position 393 | 0 minutes | 90 minutes | after 90 minutes |
| None(C) | 1 | 0.42 | 1.00 |
| E | 1 | 0.50 | 1.18 |
| K | 1 | 0.55 | 1.29 |
| D | 1 | 0.55 | 1.30 |
| Q | 1 | 0.64 | 1.52 |
| M | 1 | 0.65 | 1.53 |
| P | 1 | 0.65 | 1.54 |
| I | 1 | 0.67 | 1.58 |
| Y | 1 | 0.68 | 1.61 |
| W | 1 | 0.68 | 1.62 |
| G | 1 | 0.68 | 1.62 |
| F | 1 | 0.68 | 1.63 |
| V | 1 | 0.70 | 1.65 |
| H | 1 | 0.70 | 1.66 |
| T | 1 | 0.70 | 1.66 |
| L | 1 | 0.71 | 1.68 |
| R | 1 | 0.72 | 1.70 |
| S | 1 | 0.74 | 1.75 |
| A | 1 | 0.74 | 1.76 |
| N | 1 | 0.74 | 1.76 |

The test was conducted three times on Photuris pennsylvanica luciferase to obtain an average value of the measured values. The following table shows relative values at 37° C. for 5 minutes storage period relative to when the storage period of 0 minutes at 37° C. is defined as 1 and also shows residual activity ratios of the respective mutants relative to residual activity after 5 minutes without mutation defined as 1 (residual activity ratio after 5 minutes).

TABLE 6

| Mutation at | Storage period at 37° C. | | Residual activity ratio |
|---|---|---|---|
| position 390 | 0 minutes | 5 minutes | after 5 minutes |
| None (Y) | 1 | 0.66 | 1.00 |
| C | 1 | 0.62 | 0.94 |
| H | 1 | 0.75 | 1.14 |
| L | 1 | 0.75 | 1.14 |
| M | 1 | 0.76 | 1.15 |
| V | 1 | 0.77 | 1.16 |
| A | 1 | 0.81 | 1.23 |
| I | 1 | 0.87 | 1.33 |
| E | 1 | 0.87 | 1.33 |
| T | 1 | 0.90 | 1.36 |
| Q | 1 | 0.90 | 1.37 |
| D | 1 | 0.91 | 1.38 |
| F | 1 | 0.91 | 1.39 |
| K | 1 | 0.92 | 1.40 |
| S | 1 | 0.93 | 1.42 |
| G | 1 | 0.96 | 1.45 |
| R | 1 | 0.97 | 1.47 |
| N | 1 | 0.99 | 1.50 |
| P | 1 | 0.99 | 1.51 |

The test was conducted three times on Photinus pyralis luciferase to obtain an average value of the measured values. The following table shows relative values at 37° C. for 20 minutes storage period relative to when the storage period of 0 minutes at 37° C. is defined as 1 and also shows residual activity ratios of the respective mutants relative to residual activity after 20 minutes without mutation defined as 1 (residual activity ratio after 20 minutes).

TABLE 7

| Mutation at | Storage period at 37° C. | | Residual activity ratio |
|---|---|---|---|
| position 391 | 0 minutes | 20 minutes | after 20 minutes |
| None (C) | 1 | 0.226 | 1.00 |
| W | 1 | 0.237 | 1.05 |

As shown in Tables 5 to 7, it was exhibited that the heat resistance was able to be improved when the amino acid residue at the position corresponding to position 393 of SEQ ID NO 1 was substituted in Luciola cruciata luciferase, Photuris pennsylvanica luciferase, and Photinus pyralis luciferase as well.

The effect of improved thermal resistance was particularly significant in Luciola cruciata luciferase in the case where position 393 was substituted with asparagine, alanine, serine, arginine, leucine, threonine, histidine, valine (residual activity: 70% or more); or phenylalanine, glycine, tryptophan, tyrosine, isoleucine, proline, methionine, and glutamine (residual activity: 60% or more).

The effect of improved thermal resistance was particularly significant in Photuris pennsylvanica luciferase in the case where position 390 was substituted with proline, asparagine, arginine, glycine, serine, lysine, phenylalanine, aspartic acid, glutamine, and threonine (residual activity: 90% or more); glutamic acid, isoleucine, alanine (residual activity: 80% or more); or valine, methionine, leucine, or histidine (residual activity: 70% or more).

All publications, patents, and patent applications cited herein shall be incorporated herein by reference in their entirety.

1. A luciferase mutant having improved thermostability, wherein the luciferase mutant is a mutant of firefly luciferase, said mutant comprising an amino acid sequence in which the amino acid residue at the position corresponding to position 393 of SEQ ID NO 1 is substituted.

2. The luciferase mutant of claim 1, wherein the luciferase mutant is a mutant of a firefly luciferase comprising an amino acid sequence in which the amino acid residue at the position corresponding to position 393 of SEQ ID NO 1 is cysteine, wherein the luciferase mutant comprises an amino acid sequence in which the amino acid at the position is a non-acidic amino acid other than the cysteine.

3. The luciferase mutant of claim 1, wherein the luciferase mutant is a mutant of firefly luciferase, wherein the luciferase mutant comprises an amino acid sequence in which the amino acid at the position corresponding to position 393 of SEQ ID NO 1 is selected from the group consisting of leucine, proline, valine, isoleucine, histidine, methionine, alanine, phenylalanine, glutamine, tryptophan, tyrosine, serine, glycine, asparagine, lysine, threonine, and arginine.

4. The luciferase mutant of claim 1, wherein the luciferase mutant is a mutant of a firefly luciferase comprising an amino acid sequence in which the amino acid residue at the position corresponding to position 393 of SEQ ID NO 1 is tyrosine, wherein the luciferase mutant comprises an amino acid sequence in which the amino acid at the position is an amino acid other than tyrosine or cysteine.

5. The luciferase mutant according to claim 1, wherein the amino acid at the position corresponding to position 393 of SEQ ID NO 1 is selected from the group consisting of leucine, proline, valine, isoleucine, histidine, methionine, and alanine.

6. The luciferase mutant according to claim 5, wherein the amino acid at the position corresponding to position 393 of SEQ ID NO 1 is leucine, proline, or valine.

7. The luciferase mutant according to claim 1, wherein
the amino acid at the position corresponding to position 217 of SEQ ID NO 1 is leucine or isoleucine,
the amino acid at the position corresponding to position 490 of SEQ ID NO 1 is lysine, and/or
the amino acid at the position corresponding to position 252 of SEQ ID NO 1 is methionine.

8. The luciferase mutant according to claim 1, wherein the luciferase mutant comprises an amino acid sequence selected from the group consisting of the following (i) to (iii):
(i) an amino acid sequence selected from the group consisting of SEQ ID NOs 1, 3, 5, and 7;
(ii) an amino acid sequence in which one or several amino acids are substituted, deleted, or added at a position other than the position corresponding to position 393 of SEQ ID NO 1 in any of the amino acid sequences in (i); and
(iii) an amino acid sequence having a sequence identity of 70% or more over the full length with any of the amino acid sequences in (i) and having a sequence identity of 90% or more between a region consisting of an amino acid sequence of the following positions of SEQ ID NO 1: positions 4 and 5, positions 9 and 10, positions 13 and 14, positions 16 and 17, position 19, position 23, positions 25 and 26, position 28, positions 35 to 37, position 40, positions 42 and 43, position 45, position 47, position 55, position 57, position 62, position 65, positions 72 to 74, position 80, positions 83 to 86, positions 90 and 91, position 93, position 98, position 101, positions 105 and 106, position 111, positions 114 to 116, position 119, position 122, position 125, position 129, positions 131 and 132, position 137, position 141, position 151, position 153, position 155, position 159, position 162, position 164, position 169, position 183, position 190, positions 195 and 196, position 198, positions 200 to 202, positions 204 and 205, positions 208 to 210, position 212, position 214, positions 220 to 223, positions 226 and 227, positions 230 and 231, position 235, positions 237 and 238, position 240, position 242, positions 244 to 251, positions 253 to 257, positions 260 to 263, position 270, position 272, positions 275 and 276, positions 279 to 283, position 286, positions 289 to 293, position 300, position 302, positions 305 to 309, position 311, positions 313 to 324, positions 326 and 327, position 329, positions 332 and 333, position 335, positions 339 to 350, positions 353 to 355, position 358, positions 361 and 362, positions 365 and 366, positions 368 and 369, positions 374 and 375, position 377, position 380, position 382, positions 384 and 385, position 387, positions 390 and 391, position 396, position 398, position 400, position 403, position 406, positions 408 to 410, position 414, positions 418 to 420, positions 423 to 425, position 427, position 429, positions 433 and 434, positions 436 to 451, positions 453 to 455, position 457, positions 460 to 464, position 466, positions 468 to 471, position 473, positions 475 and 476, positions 479 to 483, position 485, positions 487 and 488, positions 492 and 493, position 497, position 504, positions 506 to 508, positions 511 and 512, positions 514 to 518, position 520, positions 522 and 523, positions 525 to 527, positions 529 to 531, position 533, position 538, position 543, and position 547 and a region consisting of an amino acid sequence of positions corresponding to the positions in the luciferase mutant.

9. The luciferase mutant according to claim 8, wherein the luciferase mutant comprises an amino acid sequence selected from the group consisting of the following (i) to (iii):
(i) the amino acid sequence of SEQ ID NO 1;
(ii) an amino acid sequence in which one or several amino acids are substituted, deleted, or added at positions other than the position corresponding to position 393 of SEQ ID NO 1 in the amino acid sequence in (i); and
(iii) an amino acid sequence having a sequence identity of 90% or more with the amino acid sequence in (i).

10. A polynucleotide encoding the luciferase mutant according to claim 1.

11. A vector comprising the polynucleotide according to claim 10.

12. A host cell comprising the polynucleotide according to claim 10.

13. A production method of a luciferase mutant having improved thermostability comprising a step of culturing the host cell according to claim 12.

14. A kit for detecting at least one of ATP, ADP, or AMP, comprising the luciferase mutant according to claim 1.

15. A method for detecting at least one of ATP, ADP, or AMP, comprising using the luciferase mutant according to claim 1.

16. A host cell comprising the vector according to claim 11.

17. A production method of a luciferase mutant having improved thermostability comprising a step of culturing the host cell according to claim 16.


SEQUENCE LISTING (74 sequences)

SEQ ID NO: 1 - protein, 548 aa, Luciola lateralis
MENMENDENIVYGPEPFYPIEEGSAGAQLRKYMDRYAKLGAIAFTNALTGVDYTYAEYLEKSCCLGEALKNYGLVVDGRIALCSENCEEFFIPVLAGLFIGVGVAPTNEIYTLRELVHSLGISKPTIVFSSKKGLDKVITVQKTVTAIKTIVILDSKVDYRGYQSMDNFIKKNTPQGFKGSSFKTVEVNRKEQVALIMNSSGSTGLPKGVQLTHENAVTRFSHARDPIYGNQVSPGTAILTVVPFHHGFGMFTTLGYLTCGFRIVMLTKFDEETFLKTLQDYKCSSVILVPTLFAILNRSELLDKYDLSNLVEIASGGAPLSKEIGEAVARRFNLPGVRQGYGLTETTSAIIITPEGDDKPGASGKVVPLFKAKVIDLDTKKTLGPNRRGEVCVKGPMLMKGYVDNPEATREIIDEEGWLHTGDIGYYDEEKHFFIVDRLKSLIKYKGYQVPPAELESVLLQHPNIFDAGVAGVPDPIAGELPGAVVVLEKGKSMTEKEVMDYVASQVSNAKRLRGGVRFVDEVPKGLTGKIDGKAIREILKKPVAKM

SEQ ID NO: 2 - DNA, 1647 bp, Luciola lateralis
atggaaaacatggagaacgatgaaaatattgtgtatggtcctgaaccattttaccctattgaagagggatctgctggagcacaattgcgcaagtatatggatcgatatgcaaaacttggagcaattgcttttactaacgcacttaccggtgtcgattatacgtacgccgaatacttagaaaaatcatgctgtctaggagaggctttaaagaattatggtttggttgttgatggaagaattgcgttatgcagtgaaaactgtgaagaattctttattcctgtattagccggtttatttataggtgtcggtgtggctccaactaatgagatttacactctacgtgaattggttcacagtttaggcatctctaagccaacaattgtatttagttctaaaaaaggattagataaagttataactgtacaaaaaacggtaactgctattaaaaccattgttatattggacagcaaagtggattatagaggttatcaatccatggacaactttattaaaaaaaacactccacaaggtttcaaaggatcaagttttaaaactgtagaagttaaccgcaaagaacaagttgctcttataatgaactcttcgggttcaaccggtttgccaaaaggtgtgcaacttactcatgaaaatgcagtcactagattttctcacgctagagatccaatttatggaaaccaagtttcaccaggcacggctattttaactgtagtaccattccatcatggttttggtatgtttactactttaggctatctaacttgtggttttcgtattgtcatgttaacgaaatttgacgaagagacttttttaaaaacactgcaagattacaaatgttcaagcgttattcttgtaccgactttgtttgcaattcttaatagaagtgaattactcgataaatatgatttatcaaatttagttgaaattgcatctggcggagcacctttatctaaagaaattggtgaagctgttgctagacgttttaatttaccgggtgttcgtcaaggctatggtttaacagaaacaacctctgcaattattatcacaccggaaggcgatgataaaccaggtgcttctggcaaagttgtgccattatttaaagcaaaagttatcgatcttgatactaaaaaaactttgggcccgaacagacgtggagaagtttgtgtaaagggtcctatgcttatgaaaggttatgtagataatccagaagcaacaagagaaatcatagatgaagaaggttggttgcacacaggagatattgggtattacgatgaagaaaaacatttctttatcgtggatcgtttgaagtctttaatcaaatacaaaggatatcaagtaccacctgctgaattagaatctgttcttttgcaacatccaaatatttttgatgccggcgttgctggcgttccagatcctatagctggtgagcttccgggagctgttgttgtacttgaaaaaggaaaatctatgactgaaaaagaagtaatggattacgttgctagtcaagtttcaaatgcaaaacgtttgcgtggtggtgtccgttttgtggacgaagtacctaaaggtctcactggtaaaattgacggtaaagcaattagagaaatactgaagaaaccagttgctaagatgtaa

SEQ ID NO: 3 - protein, 548 aa, Luciola cruciata [sequence omitted]

SEQ ID NO: 4 - DNA, 1647 bp, Luciola cruciata
atggaaaacatggaaaacgatgaaaatattgtagttggacctaaaccgttttaccctatcgaagagggatctgctggaacacaattacgcaaatacatggagcgatatgcaaaacttggcgcaattgcttttacaaatgcagttactggtgttgattattcttacgccgaatacttggagaaatcatgttgtctaggaaaagctttgcaaaattatggtttggttgttgatggcagaattgcgttatgcagtgaaaactgtgaagaattttttattcctgtaatagccggactgtttataggtgtaggtgttgcacccactaatgagatttacactttacgtgaactggttcacagtttaggtatctctaaaccaacaattgtatttagttctaaaaaaggcttagataaagttataacagtacagaaaacagtaactactattaaaaccattgttatactagatagcaaagttgattatcgaggatatcaatgtctggacacctttataaaaagaaacactccaccaggttttcaagcatccagtttcaaaactgtggaagttgaccgtaaagaacaagttgctcttataatgaactcttcgggttctaccggtttgccaaaaggcgtacaacttactcacgaaaatacagtcactagattttctcatgctagagatccgatttatggtaaccaagtttcaccaggcaccgctgttttaactgtcgttccattccatcatggttttggtatgttcactactctagggtatttaatttgtggttttcgtgttgtaatgttaacaaaattcgatgaagaaacatttttaaaaactctacaagattataaatgtacaagtgttattcttgtaccgaccttgtttgcaattctcaacaaaagtgaattactcaataaatacgatttgtcaaatttagttgagattgcatctggcggagcacctttatcaaaagaagttggtgaagctgttgctagacgctttaatcttcccggtgttcgtcaaggttatggtttaacagaaacaacatctgccattattattacaccagaaggagacgataaaccaggagcttctggaaaagtcgtgccgttgtttaaagcaaaagttattgatcttgataccaaaaaatctttaggtcctaacagacgtggagaagtttgtgttaaaggacctatgcttatgaaaggttatgtaaataatccagaagcaacaaaagaacttattgacgaagaaggttggctgcacaccggagatattggatattatgatgaagaaaaacatttctttattgtcgatcgtttgaagtctttaatcaaatacaaaggataccaagtaccacctgccgaattagaatccgttcttttgcaacatccatctatctttgatgctggtgttgccggcgttcctgatcctgtagctggcgagcttccaggagccgttgttgtactggaaagcggaaaaaatatgaccgaaaaagaagtaatggattatgttgcaagtcaagtttcaaatgcaaaacgtttacgtggtggtgttcgttttgtggatgaagtacctaaaggtcttactggaaaaattgacggcagagcaattagagaaatccttaagaaaccagttgctaagatgtga

SEQ ID NO: 5 - protein, 550 aa, Photinus pyralis
MEDAKNIKKGPAPFYPLEDGTAGEQLHKAMKRYALVPGTIAFTDAHIEVNITYAEYFEMSVRLAEAMKRYGLNTNHRIVVCSENSLQFFMPVLGALFIGVAVAPANDIYNERELLNSMNISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKSKL

SEQ ID NO: 6 - DNA, 1653 bp, Photinus pyralis
atggaagacgccaaaaacataaagaaaggcccggcgccattctatccgctagaggatggaaccgctggagagcaactgcataaggctatgaagagatacgccctggttcctggaacaattgcttttacagatgcacatatcgaggtgaacatcacgtacgcggaatacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgaacatttcgcagcctaccgtagtgtttgtttccaaaaaggggttgcaaaaaattttgaacgtgcaaaaaaaattaccaataatccagaaaattattatcatggattctaaaacggattaccagggatttcagtcgatgtacacgttcgtcacatctcatctacctcccggttttaatgaatacgattttgtaccagagtcctttgatcgtgacaaaacaattgcactgataatgaactcctctggatctactgggttacctaagggtgtggcccttccgcatagaactgcctgcgtcagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttttacgatcccttcaggattacaaaattcaaagtgcgttgctagtaccaaccctattttcattcttcgccaaaagcactctgattgacaaatacgatttatctaatttacacgaaattgcttctgggggcgcacctctttcgaaagaagtcggggaagcggttgcaaaacgcttccatcttccagggatacgacaaggatatgggctcactgagactacatcagctattctgattacacccgagggggatgataaaccgggcgcggtcggtaaagttgttccattttttgaagcgaaggttgtggatctggataccgggaaaacgctgggcgttaatcagagaggcgaattatgtgtcagaggacctatgattatgtccggttatgtaaacaatccggaagcgaccaacgccttgattgacaaggatggatggctacattctggagacatagcttactgggacgaagacgaacacttcttcatagttgaccgcttgaagtctttaattaaatacaaaggataccaggtggcccccgctgaattggagtcgatattgttacaacaccccaacatcttcgacgcgggcgtggcaggtcttcccgacgatgacgccggtgaacttcccgccgccgttgttgttttggagcacggaaagacgatgacggaaaaagagatcgtggattacgtcgccagtcaagtaacaaccgccaaaaagttgcgcggaggagttgtgtttgtggacgaagtaccgaaaggtcttaccggaaaactcgacgcaagaaaaatcagagagatcctcataaaggccaagaagggcggaaagtccaaattgtaa

SEQ ID NO: 7 - protein, 545 aa, Photuris pennsylvanica [sequence omitted]

SEQ ID NO: 8 - DNA, 1638 bp, Photuris pennsylvanica
atggaagataaaaatattttatatggacctgaaccatttcatcccttggctgatgggacggctggagaacagatgttttacgcattatctcggtatgcagatatttcaggatgcattgcattgacaaatgctcatacaaaagaaaatgttttatatgaagaatttttaaaattgtcgtgtcgtttagcggaaagttttaaaaagtatggattaaaacaaaacgacacaatagcggtgtgtagtgaaaatggtttgcaatttttccttcctttaattgcatcattgtatcttggaataattgcagcacctgttagtgataaatacattgaacgtgaattaatacacagtcttggtattgtaaaaccacgcataattttttgttccaagaatacttttcaaaaagtactgaatgtaaaatctaaattaaaatatgtagaaactattattatattagacttaaatgaagacttaggaggttatcaatgcctcaacaactttatttctcaaaattccgatattaatcttgacgtaaagaaatttaaaccaaattcttttaatcgagacgatcaggttgcgttggtaatgttttcttctggtacaactggtgtttctaagggagtcatgctaactcacaagaatattgttgcacgattttctcattgcaaagatcctacttttggtaacgcaattaatccaacgacagcaattttaacagtaataccattccaccatggttttggtatgactaccacattaggatactttacttgtggattccgagttgctctaatgcacacgtttgaagaaaaactatttttacaatcattacaagattataaagtggaaagtactttacttgtaccaacattaatggcattttttccaaaaagtgcgttagttgaaaagtacgatttatcgcacttaaaagaaattgcatctggtggcgcacctttatcaaaagaaattggggagatggtgaaaaaacggtttaaattaaactttgtcaggcaagggtatggattaacagaaaccacttcggctgttttaattacaccggacactgacgtcagaccgggatcaactggtaaaatagtaccatttcacgctgttaaagttgtggatcctacaacaggaaaaattttggggccaaatgaaactggagaattgtattttaaaggcgacatgataatgaaaagttattataataatgaagaagctactaaagcaattattaacaaagacggatggttgcgctctggtgatattgcttattatgacaatgatggccatttttatattgtggacaggctgaagtcattaattaaatataaaggttatcaggttgcacctgctgaaattgagggaatactcttacaacacccgtatattgttgatgccggcgttactggtataccggatgaagccgcgggcgagcttccagctgcaggtgttgtagtacagactggaaaatatctaaacgaacaaatcgtacaaaattttgtttccagtcaagtttcaacagccaaatggctacgtggtggggtgaaatttttggatgaaattcccaaaggatcaactggaaaaattgacagaaaagtgttaagacaaatgtttgaaaaacacaaatctaagctgtaa

SEQ ID NO: 9 - protein, 548 aa, Luciola lateralis
MENMENDENIVYGPEPFYPIEEGSAGAQLRKYMDRYAKLGAIAFTNALTGVDYTYAEYLEKSCCLGEALKNYGLVVDGRIALCSENCEEFFIPVLAGLFIGVGVAPTNEIYTLRELVHSLGISKPTIVFSSKKGLDKVITVQKTVTAIKTIVILDSKVDYRGYQSMDNFIKKNTPQGFKGSSFKTVEVNRKEQVALIMNSSGSTGLPKGVQLTHENLVTRFSHARDPIYGNQVSPGTAILTVVPFHHGFGMFTTLGYLTCGFRIVMLTKFDEETFLKTLQDYKCSSVILVPTLFAILNRSELLDKYDLSNLVEIASGGAPLSKEIGEAVARRFNLPGVRQGYGLTETTSAIIITPEGDDKPGASGKVVPLFKAKVIDLDTKKTLGPNRRGEVCVKGPMLMKGYVDNPEATREIIDEEGWLHTGDIGYYDEEKHFFIVDRLKSLIKYKGYQVPPAELESVLLQHPNIFDAGVAGVPDPIAGELPGAVVVLKKGKSMTEKEVMDYVASQVSNAKRLRGGVRFVDEVPKGLTGKIDGKAIREILKKPVAKM

SEQ ID NO: 10 - DNA, 1647 bp, Luciola lateralis
atggaaaacatggagaacgatgaaaatattgtgtatggtcctgaaccattttaccctattgaagagggatctgctggagcacaattgcgcaagtatatggatcgatatgcaaaacttggagcaattgcttttactaacgcacttaccggtgtcgattatacgtacgccgaatacttagaaaaatcatgctgtctaggagaggctttaaagaattatggtttggttgttgatggaagaattgcgttatgcagtgaaaactgtgaagagttctttattcctgtattagccggtttatttataggtgtcggtgtggctccaactaatgagatttacactctacgtgaattggttcacagtttaggcatctctaagccaacaattgtatttagttctaaaaaaggattagataaagttataactgtacaaaaaacggtaactgctattaaaaccattgttatattggacagcaaagtggattatagaggttatcaatccatggacaactttattaaaaaaaacactccacaaggtttcaaaggatcaagttttaaaactgtagaagttaaccgcaaagaacaagttgctcttataatgaactcttcgggttcaaccggtttgccaaaaggtgtgcaacttactcatgaaaatttggtcacgcgtttttctcacgctagagatccaatttatggaaaccaagtttcaccaggcacggctattttaactgtagtaccattccatcatggttttggtatgtttactactttaggctatctaacttgtggttttcgtattgtcatgttaacgaaatttgacgaagagacttttttaaaaacactgcaagattacaaatgttcaagcgttattcttgtaccgactttgtttgcaattcttaatagaagtgaattactcgataaatatgatttatcaaatttagttgaaattgcatctggcggagcacctttatctaaagaaattggtgaagctgttgctagacgttttaatttaccgggtgttcgtcaaggctatggtttaacagaaacaacctctgcaattattatcacaccggaaggcgatgataaaccaggtgcttctggcaaagttgtgccattatttaaagcaaaagttatcgatcttgatactaaaaaaactttgggcccgaacagacgtggagaagtttgtgtaaagggtcctatgcttatgaaaggttatgtagataatccagaagcaacaagagaaatcatagatgaagaaggttggttgcacacaggagatattgggtattacgatgaagaaaaacatttctttatcgtggatcgtttgaagtctttaatcaaatacaaaggatatcaagtaccacctgctgaattagaatctgttcttttgcaacatccaaatatttttgatgccggcgttgctggcgttccagatcctatagctggtgagcttccgggagctgttgttgtacttaagaaaggaaaatctatgactgaaaaagaagtaatggattacgttgctagtcaagtttcaaatgcaaaacgtttgcgtggtggtgtccgttttgtggacgaagtacctaaaggtctcactggtaaaattgacggtaaagcaattagagaaatactgaagaaaccagttgctaagatgtaa

SEQ ID NO: 11 - DNA, 29 bp, Artificial
  primer
aacttctccacgtctgttcgggcccaaag

SEQ ID NO: 12 - DNA, 29 bp, Artificial
  primer
agacgtggagaagttgcggtaaagggtcc

SEQ ID NO: 13 - DNA, 29 bp, Artificial
  primer
agacgtggagaagttgatgtaaagggtcc

SEQ ID NO: 14 - DNA, 29 bp, Artificial
  primer
agacgtggagaagttgaagtaaagggtcc

SEQ ID NO: 15 - DNA, 29 bp, Artificial
  primer
agacgtggagaagtttttgtaaagggtcc

SEQ ID NO: 16 - DNA, 29 bp, Artificial
  primer
agacgtggagaagttggcgtaaagggtcc

SEQ ID NO: 17 - DNA, 29 bp, Artificial
  primer
agacgtggagaagttcatgtaaagggtcc

SEQ ID NO: 18 - DNA, 29 bp, Artificial
  primer
agacgtggagaagttattgtaaagggtcc

SEQ ID NO: 19 - DNA, 29 bp, Artificial
  primer
agacgtggagaagttaaagtaaagggtcc

SEQ ID NO: 20 - DNA, 29 bp, Artificial
  primer
agacgtggagaagttctggtaaagggtcc

SEQ ID NO: 21 - DNA, 29 bp, Artificial
  primer
agacgtggagaagttatggtaaagggtcc

SEQ ID NO: 22 - DNA, 29 bp, Artificial
  primer
agacgtggagaagttaatgtaaagggtcc

SEQ ID NO: 23 - DNA, 29 bp, Artificial
  primer
agacgtggagaagttccggtaaagggtcc

SEQ ID NO: 24 - DNA, 29 bp, Artificial
  primer
agacgtggagaagttcaggtaaagggtcc

SEQ ID NO: 25 - DNA, 29 bp, Artificial
  primer
agacgtggagaagttcgcgtaaagggtcc

SEQ ID NO: 26 - DNA, 29 bp, Artificial
  primer
agacgtggagaagttagcgtaaagggtcc

SEQ ID NO: 27 - DNA, 29 bp, Artificial
  primer
agacgtggagaagttaccgtaaagggtcc

SEQ ID NO: 28 - DNA, 29 bp, Artificial
  primer
agacgtggagaagttgtggtaaagggtcc

SEQ ID NO: 29 - DNA, 29 bp, Artificial
  primer
agacgtggagaagtttgggtaaagggtcc

SEQ ID NO: 30 - DNA, 29 bp, Artificial
  primer
agacgtggagaagtttatgtaaagggtcc

SEQ ID NO: 31 - DNA, 1656 bp, Photuris pennsylvanica
atggaagacaagaatatcctgtatggtccggagccgtttcacccgctggcggatggcaccgcgggcgagcaaatgttttatgcgctgagccgttacgcggacattagcggttgcatcgcgctgaccaacgcgcacaccaaagagaacgtgctgtacgaggagttcctgaagctgagctgccgtctggcggagagcttcaagaaatatggcctgaagcagaacgacaccatcgcggtttgcagcgagaacggtctgcaattctttctgccgctgattgcgagcctgtacctgggcatcattgcggcgccggtgagcgataagtatattgagcgtgaactgatccacagcctgggcattgttaaaccgcgtatcattttctgcagcaaaaacacctttcagaaggttctgaacgtgaagagcaaactgaagtacgtggaaaccatcattatcctggacctgaacgaagatctgggtggctatcagtgcctgaacaacttcatcagccaaaacagcgacattaacctggatgttaagaaattcaagccgaacagctttaaccgtgacgatcaagttgcgctggtgatgtttagcagcggtaccaccggcgttagcaaaggtgtgatgctgacccacaagaacatcgtggcgcgtttcagccactgcaaagatccgacctttggcaacgcgattaacccgaccaccgcgattctgaccgtgatcccgttccaccacggttttggcatgatgaccaccctgggttacttcacctgcggctttcgtgttgcgctgatgcacaccttcgaggaaaaactgtttctgcagagcctgcaagactacaaggtggagagcaccctgctggttccgaccctgatggcgttctttccgaaaagcgcgctggttgagaagtatgatctgagccacctgaaagagattgcgagcggtggcgcgccgctgagcaaggagattggtgaaatggtgaagaaacgtttcaaactgaactttgttcgtcagggttatggcctgaccgaaaccaccagcgcggtgctgatcaccccggacaccgatgttcgtccgggtagcaccggcaagattgtgccgttccacgcggttaaagtggttgacccgaccaccggcaagatcctgggcccgaacgaaaccggtgaactgtactttaaaggcgatatgattatgaagagctactataacaacgaggaagcgaccaaagcgattatcaacaaggacggttggctgcgtagcggcgatatcgcgtactatgacaacgatggtcacttctatatcgtggaccgtctgaagagcctgattaaatacaagggctatcaggttgcgccggcggagattgaaggtatcctgctgcaacacccgtacatcgttgacgcgggtgtgaccggcattccggatgaagcggcgggcgagctgccggcggcgggcgtggttgtgcagaccggtaaatatctgaacgagcagatcgttcaaaacttcgtgagcagccaagttagcaccgcgaaatggctgcgtggtggcgtgaagtttctggatgaaatcccgaaaggtagcaccggcaagattgatcgtaaagttctgcgtcagatgtttgaaaagcacaagagcaagctgcatcatcaccatcaccactaa

SEQ ID NO: 32 - DNA, 29 bp, Artificial
  primer
aacttctccacgtctgttaggacctaaag

SEQ ID NO: 33 - DNA, 29 bp, Artificial
  primer
agacgtggagaagttgcggttaaaggacc

SEQ ID NO: 34 - DNA, 29 bp, Artificial
  primer
agacgtggagaagttgatgttaaaggacc

SEQ ID NO: 35 - DNA, 29 bp, Artificial
  primer
agacgtggagaagttgaagttaaaggacc

SEQ ID NO: 36 - DNA, 29 bp, Artificial
  primer
agacgtggagaagtttttgttaaaggacc

SEQ ID NO: 37 - DNA, 29 bp, Artificial
  primer
agacgtggagaagttggcgttaaaggacc

SEQ ID NO: 38 - DNA, 29 bp, Artificial
  primer
agacgtggagaagttcatgttaaaggacc

SEQ ID NO: 39 - DNA, 29 bp, Artificial
  primer
agacgtggagaagttattgttaaaggacc

SEQ ID NO: 40 - DNA, 29 bp, Artificial
  primer
agacgtggagaagttaaagttaaaggacc

SEQ ID NO: 41 - DNA, 29 bp, Artificial
  primer
agacgtggagaagttctggttaaaggacc

SEQ ID NO: 42 - DNA, 29 bp, Artificial
  primer
agacgtggagaagttatggttaaaggacc

SEQ ID NO: 43 - DNA, 29 bp, Artificial
  primer
agacgtggagaagttaatgttaaaggacc

SEQ ID NO: 44 - DNA, 29 bp, Artificial
  primer
agacgtggagaagttccggttaaaggacc

SEQ ID NO: 45 - DNA, 29 bp, Artificial
  primer
agacgtggagaagttcaggttaaaggacc

SEQ ID NO: 46 - DNA, 29 bp, Artificial
  primer
agacgtggagaagttcgcgttaaaggacc

SEQ ID NO: 47 - DNA, 29 bp, Artificial
  primer
agacgtggagaagttagcgttaaaggacc

SEQ ID NO: 48 - DNA, 29 bp, Artificial
  primer
agacgtggagaagttaccgttaaaggacc

SEQ ID NO: 49 - DNA, 29 bp, Artificial
  primer
agacgtggagaagttgtggttaaaggacc

SEQ ID NO: 50 - DNA, 29 bp, Artificial
  primer
agacgtggagaagtttgggttaaaggacc

SEQ ID NO: 51 - DNA, 29 bp, Artificial
  primer
agacgtggagaagtttatgttaaaggacc

SEQ ID NO: 52 - DNA, 28 bp, Artificial
  primer
cagttcaccggtttcgttcgggcccagg

SEQ ID NO: 53 - DNA, 28 bp, Artificial
  primer
gaaaccggtgaactggcgtttaaaggcg

SEQ ID NO: 54 - DNA, 28 bp, Artificial
  primer
gaaaccggtgaactggaatttaaaggcg

SEQ ID NO: 55 - DNA, 28 bp, Artificial
  primer
gaaaccggtgaactgttttttaaaggcg

SEQ ID NO: 56 - DNA, 28 bp, Artificial
  primer
gaaaccggtgaactgggctttaaaggcg

SEQ ID NO: 57 - DNA, 28 bp, Artificial
  primer
gaaaccggtgaactgcattttaaaggcg

SEQ ID NO: 58 - DNA, 28 bp, Artificial
  primer
gaaaccggtgaactgatttttaaaggcg

SEQ ID NO: 59 - DNA, 28 bp, Artificial
  primer
gaaaccggtgaactgaaatttaaaggcg

SEQ ID NO: 60 - DNA, 28 bp, Artificial
  primer
gaaaccggtgaactgctgtttaaaggcg

SEQ ID NO: 61 - DNA, 28 bp, Artificial
  primer
gaaaccggtgaactgatgtttaaaggcg

SEQ ID NO: 62 - DNA, 28 bp, Artificial
  primer
gaaaccggtgaactgaattttaaaggcg

SEQ ID NO: 63 - DNA, 28 bp, Artificial
  primer
gaaaccggtgaactgccgtttaaaggcg

SEQ ID NO: 64 - DNA, 28 bp, Artificial
  primer
gaaaccggtgaactgcagtttaaaggcg

SEQ ID NO: 65 - DNA, 28 bp, Artificial
  primer
gaaaccggtgaactgcgctttaaaggcg

SEQ ID NO: 66 - DNA, 28 bp, Artificial
  primer
gaaaccggtgaactgagctttaaaggcg

SEQ ID NO: 67 - DNA, 28 bp, Artificial
  primer
gaaaccggtgaactgacctttaaaggcg

SEQ ID NO: 68 - DNA, 28 bp, Artificial
  primer
gaaaccggtgaactggtgtttaaaggcg

SEQ ID NO: 69 - DNA, 28 bp, Artificial
  primer
gaaaccggtgaactgtgctttaaaggcg

SEQ ID NO: 70 - DNA, 28 bp, Artificial
  primer
gaaaccggtgaactggattttaaaggcg

SEQ ID NO: 71 - DNA, 28 bp, Artificial
  primer
cgcctttaaaatccagttcaccggtttc

SEQ ID NO: 72 - DNA, 29 bp, Artificial
  primer
cagagaggcgaattatgggtcagaggacc

SEQ ID NO: 73 - DNA, 29 bp, Artificial
  primer
taattcgcctctctgattaacgcccagcg

SEQ ID NO: 74 - DNA, 1665 bp, Luciola cruciata
atggaaaacatggaaaacgatgaaaatattgtagttggacctaaaccgttttaccctatcgaagagggatctgctggaacacaattacgcaaatacatggagcgatatgcaaaacttggcgcaattgcttttacaaatgcagttactggtgttgattattcttacgccgaatacttggagaaatcatgttgtctaggaaaagctttgcaaaattatggtttggttgttgatggcagaattgcgttatgcagtgaaaactgtgaagaattttttattcctgtaatagccggactgtttataggtgtaggtgttgcacccactaatgagatttacactttacgtgaactggttcacagtttaggtatctctaaaccaacaattgtatttagttctaaaaaaggcttagataaagttataacagtacagaaaacagtaactactattaaaaccattgttatattagatagtaaagttgattatcgaggatatcaatgtctggacacctttataaaaagaaacactccaccaggttttcaagcatccagtttcaaaactgtggaagttgaccgtaaagaacaagttgctcttataatgaactcttcgggttctaccggtttgccaaaaggcgtacaacttactcacgaaaatatagtcactagattttctcatgctagagatccgatttatggtaaccaagtttcaccaggcaccgctgttttaactgtcgttccattccatcatggttttggtatgttcactactctagggtatttaatttgtggttttcgtgttgtaatgttaacaaaattcgatgaagaaacatttttaaaaactctacaagattataaatgtacaagtgttattcttgtaccgaccttgtttgcaattctcaacaaaagtgaattactcaataaatacgatttgtcaaatttagttgagattgcatctggcggagcacctttatcaaaagaagttggtgaagctgttgctagacgctttaatcttcccggtgttcgtcaaggttatggtttaacagaaacaacatctgccattattattacaccggaaggtgacgataaaccaggagcttctggaaaagtcgtgccgttgtttaaagcaaaagttattgatcttgataccaaaaaatctttaggtcctaacagacgtggagaagtttgtgttaaaggacctatgcttatgaaaggttatgtaaataatccagaagcaacaaaagaacttattgacgaagaaggttggctgcacaccggagatattggatattatgatgaagaaaaacatttctttattgtcgatcgtttgaagtctttaatcaaatacaaaggataccaagtaccacctgccgaattagaatccgttcttttgcaacatccatctatctttgatgctggtgttgccggcgttcctgatcctgtagctggcgagcttccaggagccgttgttgtactggaaagcggaaaaaatatgaccgaaaaagaagtaatggattatgttgcaagtcaagtttcaaatgcaaaacgtttacgtggtggtgttcgttttgtggatgaagtacctaaaggtcttactggaaaaattgacggcagagcaattagagaaatccttaagaaaccagttgctaagatgcatcatcaccatcaccactga